(19) United States
(12) Patent Application Publication
(10) Pub. No.: US 2012/0316200 A1
(43) Pub. Date: Dec. 13, 2012

(54) PYRIDONE DERIVATIVES

(75) Inventors: Linus S. Lin, Shanghai (CN); Richard Soll, Shanghai (CN); Jingchao Dong, Shanghai (CN); Hao Wu, Shanghai (CN); Takao Suzuki, Shanghai (CN); Bin Hu, Shanghai (CN); Dejun Liu, Shanghai (CN); Jinglai Hao, Shanghai (CN); Ming Xu, Shanghai (CN)

(73) Assignee: Merck Sharp & Dohme Corp., Rahway, NJ (US)

(21) Appl. No.: 13/578,653

(22) PCT Filed: Apr. 7, 2011
(86) PCT No.: PCT/US11/31518

§ 371 Date: Aug. 13, 2012

Related U.S. Application Data
(60) Provisional application No. 61/327,881, filed on Apr. 26, 2010.

(30) Foreign Application Priority Data
Apr. 12, 2010 (CN) PCT/CN2010/071699

(51) Int. Cl. C07D 213/69; C07D 401/10; A61K 31/4709; C07D 401/12; A61P 25/22; A61P 3/04; A61P 3/10; A61P 1/16; A61P 25/24; A61K 31/4418; A61K 31/444

(52) U.S. Cl. 514/314; 546/296; 514/348; 546/167; 546/261; 514/335

(57) ABSTRACT

The present invention is directed to novel phenylpyridone derivative compounds. The compounds act as a melanin concentrating hormone receptor antagonists, and can be useful in preventing, treating or acting as a remedial agent for various circular system diseases, nervous system diseases, metabolic diseases, genital diseases, respiratory diseases and digestive diseases.

### TECHNICAL FIELD

The present invention is directed to novel pyridone derivative compounds. The compounds act as melanin concentrating hormone receptor antagonists, and can be useful in preventing, treating or acting as a remedial agent for various circular system diseases, nervous system diseases, metabolic diseases, genital diseases, respiratory diseases and digestive diseases.

### BACKGROUND

Melanin concentrating hormone (hereafter referred to as “MCH”) is a cyclic peptide hormone/neuro-peptide, which was isolated for the first time by Kawauchi, et al., in 1983. [Nature, Vol. 305, 321 (1983)]. In fish, the hormone is known to functionally antagonize melanin cell stimulating hormone, concentrate melanin granules in melanophore and participate in body color change [International Review of Cytology, Vol. 126, 1 (1991); Trends in Endocrinology and Metabolism, Vol. 5, 120 (1994)].

In mammals, MCH-containing neuron cells are localized in specific areas in the brain such as the hypothalamus lateral field, with nerve fibers projecting over a very wide scope in the brain [see The Journal of Comparative Neurology, Vol. 319, 218 (1992)]. The hypothalamus lateral field is known as the feeding center, and recent molecular biological and pharmacological discoveries suggest MCH participates in controlling energetic homeostasis. That is, it has been reported that expression of mRNA, which is an MCH precursor, is accelerated in the brains of ob/ob mice, db/db mice, Ay/a mice, Zucker fatty rats and in the brains of fasting mice [see Nature, Vol. 380, 243 (1996); Diabetes, Vol. 47, 294 (1998); Biochemical and Biophysical Research Communications, Vol. 268, 88 (2000); Molecular Brain Research, Vol. 92, 43 (2001)].

Acute ventricular administration of MCH to rats induced accelerated feeding activity [Nature, Vol. 380, 243 (1996)] and chronic administration induced obesity accompanied by polyphagy [see Proceedings of the National Academy of Sciences of the United States of America, Vol. 99, 3240 (2002)]. Moreover, MCH precursor gene-deficient mice showed ether reduced food ingestion or an increase in oxygen consumption per body weight compared to wild type mice and lower body weight due to a decrease in body fat was observed [see Nature, Vol. 396, 670 (1998)].

On the contrary, transgenic mice which express excessive MCH precursor develop obesity accompanied by polyphagy and insulin resistance [see The Journal of Clinical Investigation, Vol. 107, 379 (2001)]. Consequently, it is suggested that MCH is an important factor in developing obesity and participates in diseases induced by metabolic disorders or respiratory diseases for which obesity is one risk factor. MCH is known to participate also in anxiety-causing action, epilepsy, memory, learning, diuretic action, sodium/potassium excretory action, oxytocin secreting action, reproduction and reproductive function [see Peptides, Vol. 17, 171 (1996); Peptides, Vol. 18, 1095 (1997); Peptides, Vol. 15, 757 (1994); Journal of Neuroendocrinology, Vol. 8, 57 (1996); Critical Reviews in Neurobiology, Vol. 8, 221 (1994)].

MCH causes versatile pharmacological actions through MCH receptors which are present mainly in the central nervous system. At least two types of type 1 MCH receptors (MCH-1R or SLC-1) and type 2 MCH receptors (MCH-2R or SLT) are known [see Nature, Vol. 400, 261 (1999); Nature, Vol. 400, 265 (1999); Biochemical and Biophysical Research Communications, Vol. 261, 622 (1999); Nature Cell Biology, Vol. 1, 267 (1999); FEBS Letters, Vol. 457, 522 (1999); Biochemical and Biophysical Research Communications, Vol. 283, 1013 (2001); The Journal of Biological Chemistry, Vol. 276, 20125 (2001); Proceedings of the National Academy of Sciences of the United States of America, Vol. 98, 7564 (2001); Proceedings of the National Academy of Sciences of the United States of America, Vol. 98, 7576 (2001); The Journal of Biological Chemistry, Vol. 276, 34664 (2001); Molecular Pharmacology, Vol. 60, 632 (2001)].

The pharmacological action observed on rodents is induced mainly via MCH-1R [see Genomics, Vol. 79, 785 (2002)]. It has been observed that MCH-1R gene-deficient mice chronically administered with MCH do not develop polyphagy or obesity. Furthermore, the deficiency of MCH-1R is known to promote activity in mice [see Proceedings of the National Academy of Sciences of the United States of America, Vol. 99, 3240 (2002)], and MCH-1R's participation in central diseases accompanied by behavioral disorders, for example, attention-deficit hyperactivity disorder, schizophrenia, depression and the like also is strongly suggested [see Molecular Medicine Today, Vol. 6, 43 (2000); Trends in Neuroscience, Vol. 24, 527 (2001)].

It is also reported that an autoantibody to MCH-1R is present in serum of vitiligo vulgaris patients [see The Journal of Clinical Investigation, Vol. 109, 923 (2002)]. Furthermore, expression of MCH-1R in certain species of cancer cells was reported, and in vivo expression sites of MCH and MCH-1R also suggest MCH's participation in cancer, sleep, vigil, drug dependence and digestive disorders [see Biochemical and Biophysical Research Communications, Vol. 289, 44 (2001); Neuroendocrinology, Vol. 61, 348 (1995); Endocrinology, Vol. 137, 561 (1996); The Journal of Comparative Neurology, Vol. 435, 26 (2001)].

Functions of MCH are expressed upon it binding to MCH receptors. Therefore, when the receptor's binding to MCH receptor is inhibited, the expression of MCH action can be inhibited. In consequence, substances which are antagonists for binding of MCH with its receptor are useful for preventing, treating or acting as remedial agents for those various diseases in which MCH participates, for example, metabolic disorders such as obesity, diabetes, hormone disorder, hyperlipidemia, gout, fatty liver; cardiovascular disorders such as stenocardia, acute or congestive heart failure, myocardial infarction, coronary atherosclerosis, hypertension, renal diseases, electrolyte abnormality; central and peripheral nervous system disorders such as bulimia, emotional disturbance, depression, anxiety, epilepsy, delirium, dementia, schizophrenia, attention-deficit hyperactivity disorder, memory impairment, sleep disorders, cognitive failure, dyskinesia, paresthesias, smell disorders, morphine tolerance, drug dependence, alcoholism; reproductive disorders such as infertility, preterm labor and sexual dysfunction; and other digestive disorders, respiratory disorders, cancer or pigmentation.

### SUMMARY OF THE INVENTION

The present invention is directed to pyridone derivative compounds of formula I

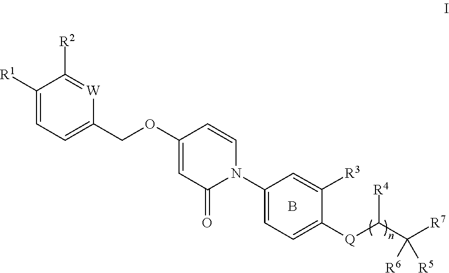

having neutral functionalities. Such compounds are antagonists of the melanin-concentrating hormone (“MCH”) type 1 receptor which are useful in the treatment or prevention of diseases in which the melanin-concentrating hormone is involved, such as metabolic diseases such as obesity, diabetes, hormone secretion disorder, hyperlipemia, gout and fatty liver; circulatory diseases such as angina pectoris, acute/congestive cardiac insufficiency, myocardial infarction, coronary arteriosclerosis, hypertension, nephropathy and electrolyte abnormality; central and peripheral nervous system diseases such as bulimia, affective disorder, depression, anxiety, epilepsy, delirium, dementia, schizophrenia, attention deficit/hyperactivity disorder, dysmnesia, somnipathy, cognitive impairment, dyskinesia, dysesthesia, dysosmia, morphine resistance, drug dependence and alcohol dependence; reproductive system diseases such as infertility, premature delivery and sexual dysfunction; and other conditions including digestive diseases, respiratory diseases, cancer, chromatosis. The invention also provides a pharmaceutical composition comprising a pharmaceutically acceptable carrier and at least one of compounds described herein.

### DETAILED DESCRIPTION

### Compounds

The compounds described herein are antagonists of the MCH receptor and are effective in the prevention or treatment of various MCH-receptor-related diseases. Compounds of the present invention are described by structural formula I:

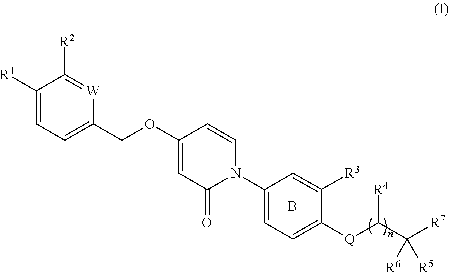

and pharmaceutically acceptable salts thereof, wherein R¹ and R² are independently selected from the group consisting of halogen, hydrogen, —OH, C₁-C₆alkyl, —OC₁-C₆alkyl, —O-halogen-substitutedC₁-C₆ alkyl and halogen-substitutedC₁-C₆ alkyl; W is —N— or —C—; Q is —O—, —NH—, or —C—; R³ is a halogen, hydrogen, —OC₁-C₆alkyl, C₁-C₆alkyl, —O-halogen-substitutedC₁-C₆alkyl, halogen-substitutedC₁-C₆alkyl, cyano, —SO₂C₁-C₆alkyl or taken together with aromatic ring B, R⁴ and Q form a heteroaryl; R⁴ is hydrogen, oxo, C₁-C₆alkyl, halogen-substitutedC₁-C₆alkyl, or together with aromatic ring B, R³ and Q form a heteroaryl or taken together with R⁵ form C₃-C₆cycloalkyl; R⁵, R⁶ and R⁷ are each independently selected from the group consisting of hydrogen, C₁-C₆alkyl, C₃-C₆cycloalkyl, C₁-C₆alkylC₃-C₆cycloalkyl, —OH, C₁-C₆alkyl-OH and —OC₁-C₆alkyl, halogen-substitutedC₁-C₆alkyl, halogen-substitutedC₃-C₆alkyl or when R⁵ and R⁶ are taken together form an oxo group or C₃-C₆cycloalkyl, or when R⁵ and R⁴ taken together form a C₃-C₆cycloalkyl; and n is 1-3.

In certain embodiments of the compounds described herein, R¹ is selected from the group consisting of halogen, hydrogen, —OH, C₁-C₆alkyl, —OC₁-C₆alkyl, —O-halogen-substitutedC₁-C₆ alkyl and halogen-substitutedC₁-C₆ alkyl. In some embodiments of the compounds described herein, R¹ is halogen or hydrogen. In other embodiments, R¹ is hydrogen. In still other embodiments, R¹ is a halogen wherein the halogen is selected from fluorine or chlorine. In certain embodiments of the compounds described herein, R² is selected from the group consisting of halogen, hydrogen, —OH, C₁-C₆alkyl, —OC₁-C₆alkyl, —O-halogen-substitutedC₁-C₆ alkyl and halogen-substitutedC₁-C₆ alkyl. For example, in some embodiments of the compounds described herein, R² is halogen or hydrogen. In other embodiments, R² is hydrogen. In still other embodiments, R² is a halogen wherein the halogen is selected from fluorine or chlorine.

In certain embodiments of the compounds described herein, R¹ and R² are both halogen. In other embodiments of the compounds described herein, R¹ and R² are both hydrogen. In still other embodiments of the compounds described herein, R¹ is a halogen and R² is hydrogen. In yet other embodiments of the compounds described herein, R¹ is hydrogen and R² is halogen.

For example, in one embodiment R¹ and R² are halogen, wherein the halogen is selected from the group consisting of fluorine and chlorine. Additionally, in another example R¹ is halogen, wherein the halogen is selected from the group consisting of fluorine and chlorine and R² is hydrogen. Alternatively, in another example R² is halogen, wherein the halogen is selected from the group consisting of fluorine and chlorine and R¹ is hydrogen.

In certain embodiments of the compounds described herein, W is —N—. In other embodiments of the compounds described herein, W is —C—.

In certain embodiments of the compounds described herein, Q is selected from the group consisting of —O—, —NH—, and —C—. In some embodiments described herein Q is —O— or —C—. For example, in certain embodiments, Q is —O—. In other embodiments, Q is —C—. In yet other embodiments, Q is —NH—. In still other embodiments, Q is —O— or —C— or Q is only —NH— when Q, taken together with R⁴, aromatic ring B and R³ form a heteroaryl.

In certain embodiments of the compounds described herein, R³ is a halogen, hydrogen, —OC₁-C₆alkyl, C₁-C₆alkyl, —O-halogen-substitutedC₁-C₆alkyl, halogen-substitutedC₁-C₆alkyl, cyano, —SO₂C₁-C₆alkyl or taken together with aromatic ring B, R⁴ and Q form a heteroaryl. For example, in some embodiments, R³ is a halogen, hydrogen, —OC₁-C₆alkyl, C₁-C₆alkyl. In other embodiments, R³ is hydrogen or —OC₁-C₆alkyl. In still other embodiments, R³ is —OC₁-C₆alkyl. In another embodiment of the compounds described herein, R³ is hydrogen. In another embodiment, R³ is a halogen selected from the group consisting of chlorine or fluorine. In another embodiment, R³ is —O-halogen-substitutedC₁-C₆alkyl or halogen-substitutedC₁-C₆alkyl, wherein the halogen is selected from the group consisting of chlorine or fluorine. In still other embodiments, R³ is cyano. In yet other embodiments, R³ is —SO₂C₁-C₆alkyl such as methanesulfonyl.

In certain embodiments of the compounds described herein, R⁴ is hydrogen, oxo, halogen-substitutedC₁-C₆alkyl, C₁-C₆alkyl or together with aromatic ring B, R³ and Q form a heteroaryl or taken together with R⁵ form a C₃-C₆cycloalkyl. In certain embodiments, R⁴ is an oxo group. In certain embodiments, R⁴ is halogen-substitutedC₁-C₆alkyl. In certain embodiments, R⁴ is C₁-C₆alkyl. In other embodiments, R⁴ is hydrogen. In still other embodiments R⁴, taken together with aromatic ring B, R³ and Q, form a heteroaryl such as the following:

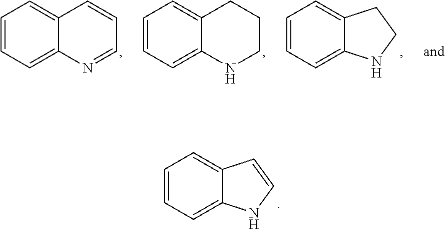

In certain embodiments of the compounds described herein, R⁵, R⁶ and R⁷ are each independently selected from the group consisting of hydrogen, C₁-C₆alkyl, C₃-C₆cycloalkyl, halogen-substitutedC₁-C₆alkyl, halogen-substitutedC₃-C₆cycloalkyl, C₁-C₆alkylC₃-C₆cycloalkyl, —OH, C₁-C₆alkyl-OH and —OC₁-C₆alkyl. In certain embodiments, R⁵, R⁶ and R⁷ are not all hydrogen at the same time. For example, in certain embodiments of the compounds described herein at least one of R⁵, R⁶ and R⁷ is not hydrogen. In certain embodiments, R⁵ and R⁶ are taken together form an oxo group or C₃-C₆cycloalkyl. In other embodiments, R⁴ and R⁵ are taken together form a C₃-C₆cycloalkyl. For example, in some embodiments, R⁵, R⁶, R⁷ are independently selected from the group consisting of hydrogen, —OH, C₁-C₆alkyl-OH and cyclopropane. In other embodiments, R⁵, R⁶, R⁷ are independently selected from the group consisting of hydrogen, —OH, C₁-C₆alkyl-OH and C₁-C₆alkylC₃-C₆cycloalkyl. In other embodiments, R⁵, R⁶, R⁷ are independently selected from the group consisting of halogen-substitutedC₁-C₆alkyl and halogen-substitutedC₃-C₆cycloalkyl.

In still other embodiments, R⁵ and R⁶ together form an oxo group and R⁷ is —OC₂-C₆alkyl. In still other embodiments, R⁵ and R⁶ are C₁-C₆alkyl and R⁷ is C₁-C₆alkyl-OH. Alternatively, in other embodiments R⁵ and R⁶ together are cyclopropyl and R⁷ is —OC₁-C₆alkyl.

In certain embodiments of the compounds described herein, n is 1-3. In some embodiments of the compounds described herein n is 1. In other embodiments described herein, n is 2. In still other embodiments, n is 3.

In certain embodiments the compounds of the present invention can be described as formula Ia:

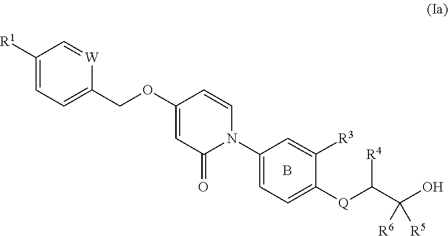

and pharmaceutically acceptable salts thereof, wherein R¹ is halogen or halogen-substitutedC₁-C₆ alkyl; W is —N— or —C—; Q is —O—, —NH— or —C—; R³ is a halogen, hydrogen, —OC₁-C₆alkyl, C₁-C₆alkyl, —O-halogen-substitutedC₁-C₆alkyl, halogen-substitutedC₁-C₆alkyl, cyano, —SO₂C₁-C₆alkyl or taken together with aromatic ring B, Q and R⁴ form a heteroaryl; R⁴ is hydrogen, oxo, C₁-C₆alkyl, halogen-substitutedC₁-C₆alkyl or together with aromatic ring B, R³ and Q form a heteroaryl, or taken together with R⁵ form a C₃-C₆cycloalkyl; R⁵ and R⁶ are each independently selected from the group consisting of hydrogen, C₁-C₆alkyl, halogen-substitutedC₁-C₆alkyl, C₃-C₆cycloalkyl, halogen-substitutedC₃-C₆cycloalkyl, C₁-C₆alkylC₃-C₆cycloalkyl, or taken together form a C₃-C₆cycloalkyl.

In certain embodiments of the compounds described by formula Ia, R¹ is selected from the group consisting of halogen and halogen-substitutedC₁-C₆ alkyl. In some embodiments of the compounds described herein R¹ is halogen, wherein the halogen is selected from fluorine or chlorine. For example, in some embodiments, R¹ is fluorine. In other embodiments, R¹ is halogen-substitutedC₁-C₆ alkyl, wherein the halogen-substitutedC₁-C₆ alkyl is selected from the group consisting of fluoromethyl, difluoromethyl and trifluoromethyl.

In certain embodiments of the compounds described by formula Ia, W is —N—. In other embodiments of the compounds described herein, W is —C—.

In certain embodiments of the compounds described by formula Ia, Q is selected from the group consisting of —O—, —NH—, and —C—. In some embodiments described herein Q is —O— or —C—. For example, in certain embodiments, Q is —O—. In other embodiments, Q is —C—. In yet other embodiments, Q is —NH—. In still other embodiments, Q is —O— or —C— or Q is only —NH— when Q, taken together with R⁴, aromatic ring B and R³ form a heteroaryl.

In certain embodiments of the compounds described by formula Ia, R³ is a halogen, hydrogen, —OC₁-C₆alkyl, C₁-C₆alkyl, —O-halogen-substitutedC₁-C₆alkyl, halogen-substitutedC₁-C₆alkyl or taken together with aromatic ring B, R⁴ and Q form a heteroaryl. For example, in some embodiments, R³ is a halogen, hydrogen, —OC₁-C₆alkyl, C₁-C₆alkyl. In other embodiments, R³ is hydrogen or —OC₁-C₆alkyl. In still other embodiments, R³ is —OC₁-C₆alkyl. In still other embodiments, R³ is —O-halogen-substitutedC₁-C₆alkyl or halogen-substitutedC₁-C₆alkyl. In another embodiment of the compounds described herein, R³ is hydrogen. In another embodiment, R³ is a halogen selected from the group consisting of chlorine or fluorine.

In certain embodiments of the compounds described as formula Ia, R⁴ is hydrogen, oxo, halogen-substitutedC₁-C₆alkyl, C₁-C₆alkyl or together with aromatic ring B, R³ and Q form a heteroaryl or taken together with R⁵ form a C₃-C₆cycloalkyl. In certain embodiments R⁴ is an oxo group. In other embodiments, R⁴ is hydrogen. In other embodiments, R⁴ is halogen-substitutedC₁-C₆alkyl or C₁-C₆alkyl. In still other embodiments R⁴, taken together with aromatic ring B, R³ and Q form a heteroaryl such as the following:

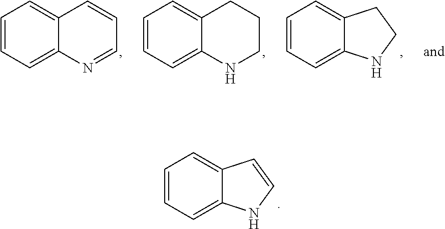

In certain embodiments of the compounds described as formula Ia, R⁵ and R⁶ are each independently selected from the group consisting of hydrogen, C₁-C₆alkyl, C₃-C₆cycloalkyl, halogen-substitutedC₁-C₆alkyl, halogen-substitutedC₃-C₆cycloalkyl, C₁-C₆alkylC₃-C₆cycloalkyl, or R⁵ and R⁶ taken together form a C₃-C₆cycloalkyl. In some embodiments, R⁵ and R⁶ are both C₁-C₆alkyl. In other embodiments, R⁵ and R⁶ are both methyl. In still other embodiments, R⁵ is hydrogen and R⁶ is cyclopropyl. In yet another embodiment, R⁵ and R⁶ together form cyclopropyl or cyclobutyl. In yet another embodiment, R⁵ and R⁶ are independently selected from the group consisting of halogen-substitutedC₁-C₆alkyl and halogen-substitutedC₃-C₆cycloalkyl.

In certain embodiments the compounds of the present invention can be described as formula Ib:

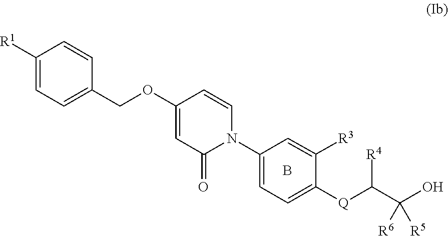

and pharmaceutically acceptable salts thereof, wherein R¹ is a halogen or halogen-substitutedC₁-C₆ alkyl; —NH— or —C—; R³ is a halogen, hydrogen, —OC₁-C₆alkyl, C₁-C₆alkyl, O-halogen-substitutedC₁-C₆alkyl, halogen-substitutedC₁-C₆alkyl, cyano, SO₂C₁-C₆alkyl or taken together with aromatic ring B, Q and R⁴ form a heteroaryl; R⁴ is hydrogen, oxo, C₁-C₆alkyl, halogen substitutedC₁-C₆alkyl or together with aromatic ring B, R³ and Q form a heteroaryl, or taken together with R⁵ form a C₃-C₆cycloalkyl; R⁵ and R⁶ are each independently selected from the group consisting of hydrogen, C₁-C₆alkyl, halogen-substitutedC₁-C₆alkyl, C₃-C₆cycloalkyl, halogen-substitutedC₃-C₆cycloalkyl, C₁-C₆alkylC₃-C₆cycloalkyl, or taken together form a C₃-C₆cycloalkyl.

In certain embodiments of the compounds described by formula Ib, R¹ is selected from the group consisting of halogen and halogen-substitutedC₁-C₆ alkyl. In some embodiments of the compounds described herein R¹ is halogen, wherein the halogen is selected from fluorine or chlorine. For example, in some embodiments, R¹ is fluorine. In other embodiments, R¹ is halogen-substitutedC₁-C₆ alkyl, wherein the halogen-substitutedC₁-C₆ alkyl is selected from the group consisting of fluoromethyl, difluoromethyl and trifluoromethyl.

In certain embodiments of the compounds described by formula Ib, Q is selected from the group consisting of —O—, —NH—, and —C—. In some embodiments described herein Q is —O— or —C—. For example, in certain embodiments, Q is —O—. In other embodiments, Q is —C—. In yet other embodiments, Q is —NH—. In still other embodiments, Q is —O— or —C— or Q is only —NH— when Q, taken together with R⁴, aromatic ring B and R³ form a heteroaryl.

In certain embodiments of the compounds described by formula Ib, R³ is a halogen, hydrogen, —OC₁-C₆alkyl, C₁-C₆alkyl, —O-halogen-substitutedC₁-C₆alkyl, halogen-substitutedC₁-C₆alkyl or taken together with aromatic ring B, R⁴ and Q form a heteroaryl. For example, in some embodiments, R³ is a halogen, hydrogen, —OC₁-C₆alkyl, C₁-C₆alkyl. In other embodiments, R³ is hydrogen or —OC₁-C₆alkyl. In still other embodiments, R³ is —OC₁-C₆alkyl. In still other embodiments, R³ is —O-halogen-substitutedC₁-C₆alkyl or halogen-substitutedC₁-C₆alkyl. In another embodiment of the compounds described herein, R³ is hydrogen. In another embodiment, R³ is a halogen selected from the group consisting of chlorine or fluorine.

In certain embodiments of the compounds described as formula Ib, R⁴ is hydrogen, oxo, halogen-substitutedC₁-C₆alkyl, C₁-C₆alkyl or together with aromatic ring B, R³ and Q form a heteroaryl or taken together with R⁵ form a C₃-C₆cycloalkyl. In certain embodiments R⁴ is an oxo group. In other embodiments, R⁴ is hydrogen. In other embodiments, R⁴ is halogen-substitutedC₁-C₆alkyl or C₁-C₆alkyl. In still other embodiments R⁴, taken together with aromatic ring B, R³ and Q form a heteroaryl such as the following:

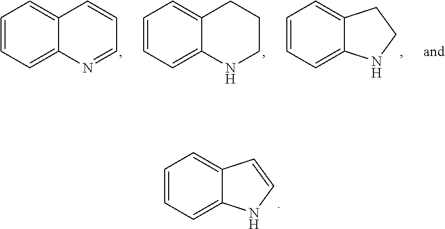

In certain embodiments of the compounds described as formula Ib, R⁵ and R⁶ are each independently selected from the group consisting of hydrogen, C₁-C₆alkyl, C₃-C₆cycloalkyl, halogen-substitutedC₁-C₆alkyl, halogen-substitutedC₃-C₆cycloalkyl, C₁-C₆alkylC₃-C₆cycloalkyl, or taken together form a C₃-C₆cycloalkyl. In some embodiments, R⁵ and R⁶ are both C₁-C₆alkyl. In other embodiments, R⁵ and R⁶ are both methyl. In still other embodiments, R⁵ is hydrogen and R⁶ is cyclopropyl. In yet another embodiment, R⁵ and R⁶ together form cyclopropyl or cyclobutyl. In yet another embodiment, R⁵ and R⁶ are independently selected from the group consisting of halogen-substitutedC₁-C₆alkyl and halogen-substitutedC₃-C₆cycloalkyl.

In any of the embodiments above, if not already specified, C₁-C₆alkyl, C₃-C₆cycloalkyl, —OC₁-C₆alkyl, C₁-C₆alkylC₃-C₆cycloalkyl can further be substituted with one or more halogens.

Non-limiting examples of the compounds encompassed by the present invention include the examples shown in Table 1:

TABLE 1

| | |
|---|---|
| 4-[(4-fluorobenzyl)oxy]-1-[4-(2-hydroxy-2- methylpropoxy)-3-methylphenyl]pyridin- 2(1H)-one | 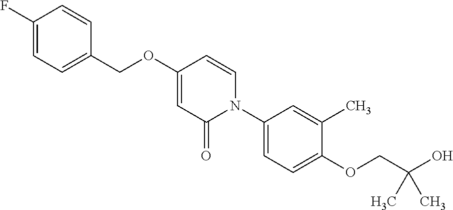 |
| 1-[4-(2-cyclopropyl-2- hydroxyethoxy)phenyl]-4-[(4- fluorobenzyl)oxy]pyridin-2(1H)-one | 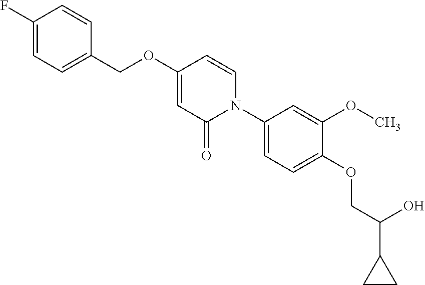 |
| 1-[4-(2-cyclopropyl-2-hydroxyethoxy)-3- methoxyphenyl]-4-[(4- fluorobenzyl)oxy]pyridin-2(1H)-one | 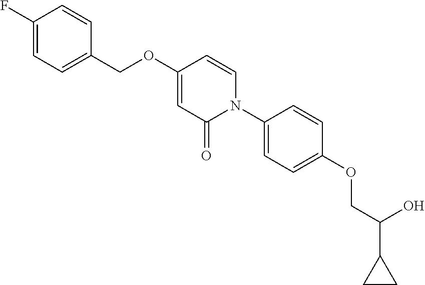 |
| 1-[3-chloro-4-(2-hydroxy-2- methylpropoxy)phenyl]-4-[(4- fluorobenzyl)oxy]pyridin-2(1H)-one | 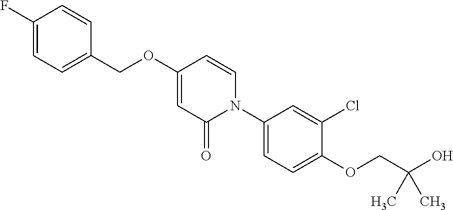 |
| 4-[(4-fluorobenzyl)oxy]-1-[4-(2-hydroxy-2- methylpropoxy)phenyl]pyridin-2(1H)-one | 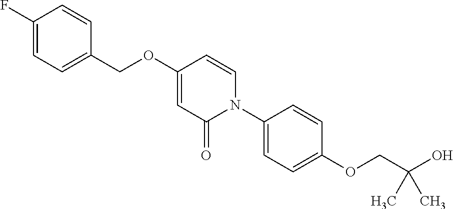 |
| 4-[(4-fluorobenzyl)oxy]-1-[4-(2-hydroxy-2- methylpropoxy)-3-methoxyphenyl]pyridin- 2(1H)-one | 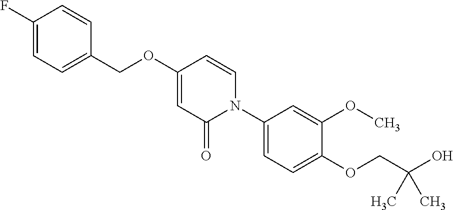 |
| 4-[(4-fluorobenzyl)oxy]-1-[3-fluoro-4-(2- hydroxy-2-methylpropoxy)phenyl]pyridin- 2(1H)-one | 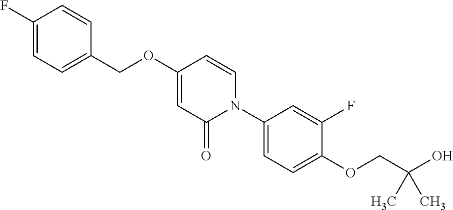 |
| 4-[(4-fluorobenzyl)oxy]-1-[4-(3-hydroxy-2,2- dimethylpropoxy)phenyl]pyridin-2(1H)-one | 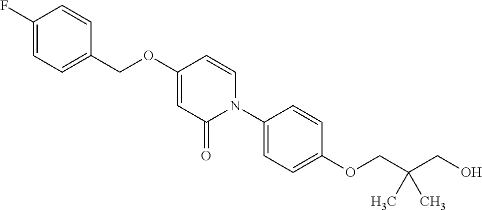 |
| 4-[(4-fluorobenzyl)oxy]-1-[4-(3-hydroxy-2,2- dimethylpropoxy)-3-methoxyphenyl]pyridin- 2(1H)-one | 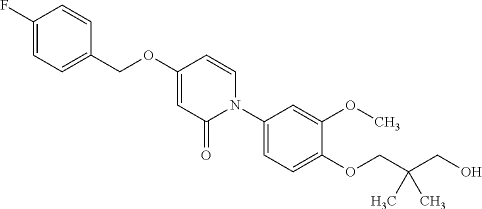 |
| 4-(4-Fluorobenzyloxy)-1-[2-(2- hydroxypropan-2-yl)quinolin-6-yl]pyridin-2- (1H)-one | 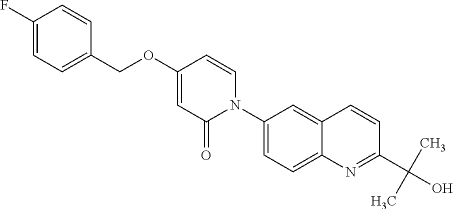 |
| 4-[(5-chloropyridin-2-yl)methoxy]-1-[4-(2- cyclopropyl-2-hydroxyethoxy)-3- methoxyphenyl]pyridin-2(1H)-one | 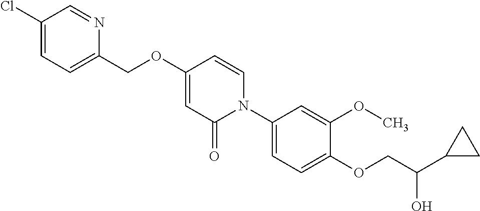 |
| 4-[(5-chloropyridin-2-yl)methoxy]-1-[4-(2- cyclopropyl-2-hydroxyethoxy)phenyl]pyridin- 2(1H)-one | 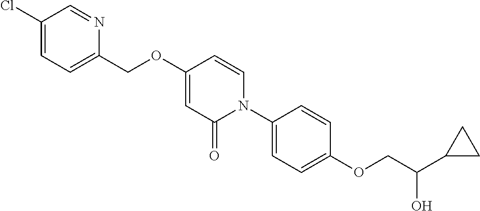 |
| 1-[4-(2-cyclopropyl-2- hydroxyethoxy)phenyl]-4-[(3,4- difluorobenzyl)oxy]pyridin-2(1H)-one | 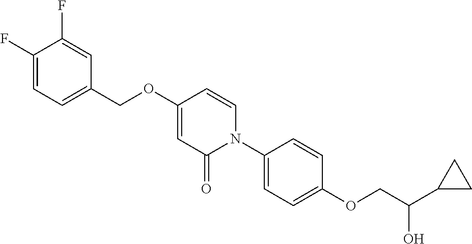 |
| 1-{4-[(2R)-2-cyclopropyl-2-hydroxyethoxy]- 3-methoxyphenyl}-4-[(4- fluorobenzyl)oxy]pyridin-2(1H)-one | 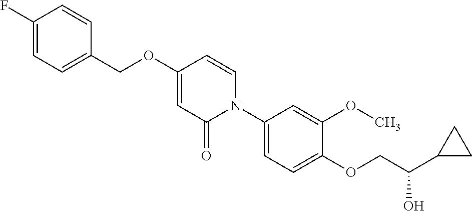 |
| 1-{4-[(2R)-2-cyclopropyl-2-hydroxyethoxy]- 3-methoxyphenyl}-4-[(4- fluorobenzyl)oxy]pyridin-2(1H)-one | 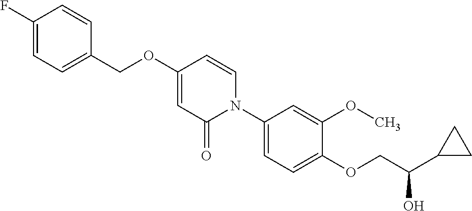 |
| 1-{4-[(2S)-2-cyclopropyl-2- hydroxyethoxy]phenyl}-4-[(4- fluorobenzyl)oxy]pyridin-2(1H)-one | 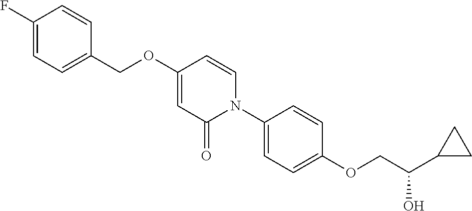 |
| 1-{4-[(2R)-2-cyclopropyl-2- hydroxyethoxy]phenyl}-4-[(4- fluorobenzyl)oxy]pyridin-2(1H)-one | 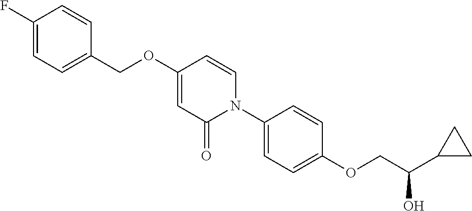 |
| methyl (2-fluoro-4-{4-[(4-fluorobenzyl)oxy]- 2-oxopyridin-1(2H)-yl}phenoxy)acetate | 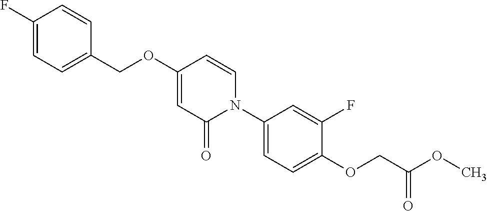 |
| 4-[(4-fluorobenzyl)oxy]-1-{4-[(1- hydroxycyclopropyl)methoxy]-3- methylphenyl}pyridin-2(1H)-one | 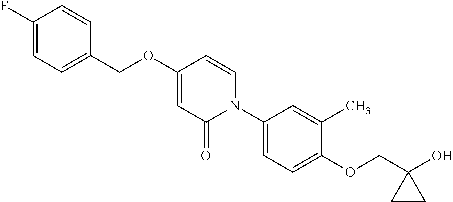 |
| propan-2-yl (4-{4-[(4-fluorobenzyl)oxy]-2- oxopyridin-1(2H)-yl}-2- methylphenoxy)acetate | 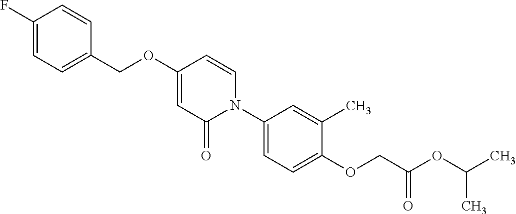 |
| methyl (4-{4-[(4-fluorobenzyl)oxy]-2- oxopyridin-1(2H)-yl}-2- methylphenoxy)acetate | 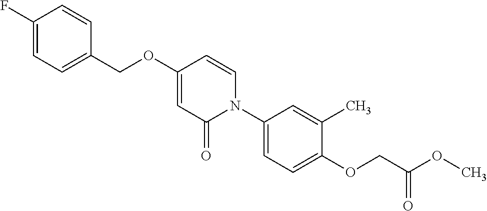 |
| methyl (4-{4-[(4-fluorobenzyl)oxy]-2- oxopyridin-1(2H)-yl}-2- methoxyphenoxy)acetate | 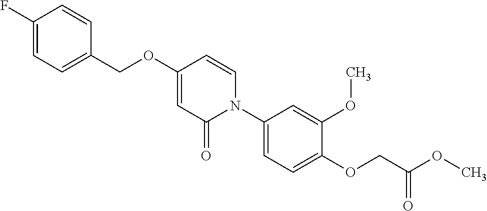 |
| 4-[(4-fluorobenzyl)oxy]-1-[4-(3-hydroxy-3- methylbutoxy)-3-methoxyphenyl]pyridin- 2(1H)-one | 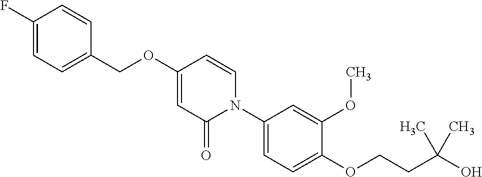 |
| 4-[(5-chloropyridin-2-yl)methoxy]-1-{4- [(2R)-2-cyclopropyl-2-hydroxyethoxy]-3- methoxyphenyl}pyridin-2(1H)-one | 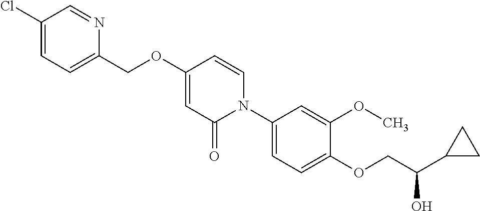 |
| 4-[(5-chloropyridin-2-yl)methoxy[-1-{4- [(2R)-2-cyclopropyl-2- hydroxyethoxy]phenyl}pyridin-2(1H)-one | 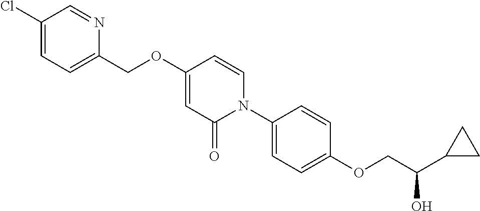 |
| 1-{4-[(2R)-2-cyclopropyl-2- hydroxyethoxy]phenyl}-4-[(3,4- difluorobenzyl)oxy]pyridin-2(1H)-one | 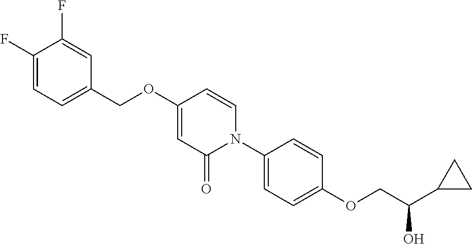 |
| 4-[(5-chloropyridin-2-yl)methoxy]-1-{4- [(2S)-2-cyclopropyl-2-hydroxyethoxy]-3- methoxyphenyl}pyridin-2(1H)-one | 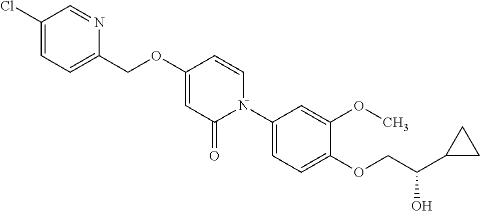 |
| 1-{4-[(2S)-2-cyclopropyl-2- hydroxyethoxy]phenyl}-4-[(3,4- difluorobenzyl)oxy]pyridin-2(1H)-one | 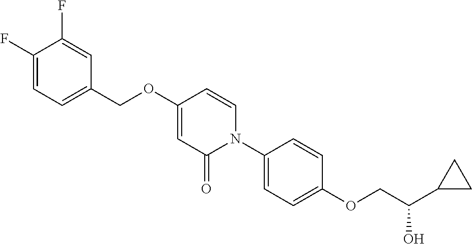 |
| 4-[(5-chloropyridin-2-yl)methoxy]-1-{4- [(2S)-2-cyclopropyl-2- hydroxyethoxy]phenyl}pyridin-2(1H)-one | 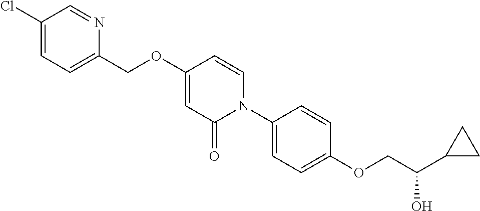 |
| 1-{3-chloro-4-[(1- hydroxycyclopropyl)methoxy]phenyl}-4-[(4- fluorobenzyl)oxy]pyridin-2(1H)-one | 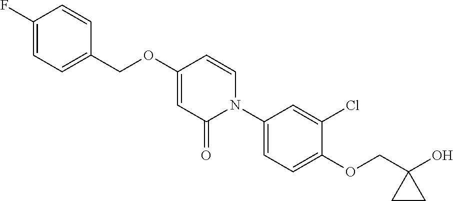 |
| 4-[(4-fluorobenzyl)oxy]-1-{3-fluoro-4-[(1- hydroxycyclopropyl)methoxy]phenyl}pyridin- 2(1H)-one | 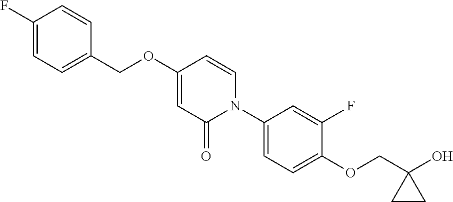 |
| 4-[(4-fluorobenzyl)oxy]-1-{4-[(1- hydroxycyclopropyl)methoxy]-3- methoxyphenyl}pyridin-2(1H)-one | 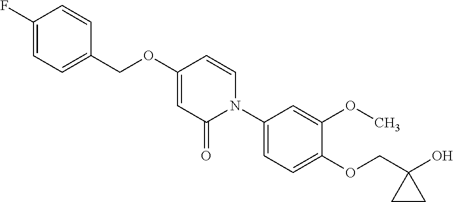 |
| 1-{4-[(2S)-2-cyclopropyl-2-hydroxyethoxy]- 3-methoxyphenyl}-4-[(3,4- difluorobenzyl)oxy]pyridin-2(1H)-one | 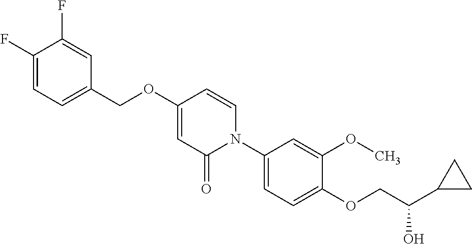 |
| 1-{4-[(2R)-2-cyclopropyl-2-hydroxyethoxy]- 3-methoxyphenyl}-4-[(3,4- difluorobenzyl)oxy]pyridin-2(1H)-one | 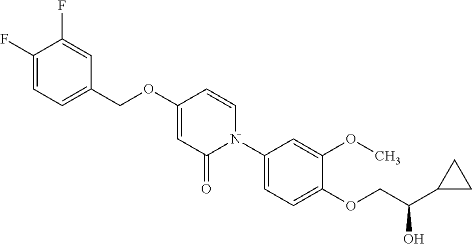 |

For example, in some embodiments the compounds described herein are:
4-[(4-Fluorobenzyloxy)]-1-[4-(2-hydroxy-2-methylpropoxy)-3-methylphenyl]pyridin-2(1H)-one;
4-[(4-Fluorobenzyl)oxy]-1-{4-[(1-hydroxycyclopropyl)methoxy]-3-methylphenyl}pyridin-2(1H)-one;
4-(4-Fluorobenzyloxy)-1-[2-(2-hydroxypropan-2-yl)quinolin-6-yl]pyridin-2-(1H)-one;
1-[3-chloro-4-(2-hydroxy-2-methylpropoxy)phenyl]-4-[(4-fluorobenzyl)oxy]pyridin-2(1H)-one;
4-[(5-chloropyridin-2-yl)methoxy]-1-[4-(2-cyclopropyl-2-hydroxyethoxy)phenyl]pyridin-2(1H)-one;
4-[(5-chloropyridin-2-yl)methoxy]-1-{4-[(2R)-2-cyclopropyl-2-hydroxyethoxy]phenyl}pyridin-2(1H)-one;
4-[(5-chloropyridin-2-yl)methoxy]-1-{4-[(2S)-2-cyclopropyl-2-hydroxyethoxy]phenyl}pyridin-2(1H)-one; and
1-{3-chloro-4-[(1-hydroxycyclopropyl)methoxy]phenyl}-4-[(4-fluorobenzyl)oxy]pyridin-2(1H)-one.

### DEFINITIONS

Examples of “halogen” include a fluorine atom, a chlorine atom, a bromine atom, and an iodine atom.

“C₃-C₆cycloalkyl” encompasses cycloalkyls having 3 to 6 carbons, forming one or more carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“—OC1-C₆alkyl” refers to an alkyl group having 1 to 6 carbons linked to oxygen, also known as an alkoxy group.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “halogen-substitutedC₁-C₆ alkyl” encompasses C₁-C₆ alkyl with the hydrogen atoms thereof being partially or completely substituted with halogen, examples thereof including fluoromethyl, difluoromethyl, trifluoromethyl, 2-fluoroethyl, 1,2-difluoroethyl, 2,2-difluoroethyl and the like.

The term “C₁-C₆ alkyl-OH” encompasses C₁-C₆ alkyl with one or more of the hydrogen atoms thereof being substituted with —OH, also known as an alcohol group, examples thereof including methanol, ethanol, propanol and butanol.

“Heteroaryl” unless otherwise specified, means an aromatic or partially aromatic heterocycle that contains at least one ring heteroatom selected from O, S and N. Heteroaryls also include heteroaryls fused to other kinds of rings, such as aryls, cycloalkyls and heterocycles that are not aromatic. Examples of heteroaryl groups include pyrrolyl, isoxazolyl, isothiazolyl, pyrazolyl, pyridinyl, 2-oxo-(1B)-pyridinyl (2-hydroxy-pyridinyl), oxazolyl, 1,2,4-oxadiazolyl, 1,3,4-oxadiazolyl, thiadiazolyl, thiazolyl, imidazolyl, triazolyl, tetrazolyl, furyl, triazinyl, thienyl, pyrimidinyl, pyrazinyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, imidazo[1,2-a]pyridinyl, [1,2,4-triazolo][4,3-a]pyridinyl, pyrazolo[1,5-a]pyridinyl, [1,2,4-triazolo][1,5-c]pyridinyl, 2-oxo-1,3-benzoxazolyl, 4-oxo-3H-quinazolinyl, 3-oxo-[1,2,4]-triazolo[4,3-a]-2H-pyridinyl, 5-oxo-[1,2,4]-4H-oxadiazolyl, 2-oxo-[1,3,4]-3H-oxadiazolyl, 2-oxo-1,3-dihydro-2H-imidazolyl, 3-oxo-2,4-dihydro-3H-1,2,4-triazolyl, and the like. For heterocyclyl and heteroaryl groups, rings and ring systems containing from 3-15 atoms are included, forming 1-3 rings.

“Oxo” means the functional group “═O”, such as, for example, (1) “C═(O)”, that is a carbonyl group; (2) “S═(O)”, that is, a sulfoxide group; and (3) “N═(O)”, that is, an N-oxide group, such as pyridyl-N-oxide.

“SO₂C₁-C₆alkyl” group means a group in which a C₁-C₆alkyl group is attached to a sulfonyl (—SO₂—) group. Specific examples thereof include methanesulfonyl, ethanesulfonyl, n-propylsulfonyl, isopropanesulfonyl, n-butanesulfonyl, sec-butanesulfonyl and tert-butanesulfonyl groups and the like.

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mutate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangarnous, potassium, sodium, zinc, and the like, Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glutamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The compounds of the present invention contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that, as used herein, references to the compounds of the structural formulas described herein are meant to also include the pharmaceutically acceptable salts, and also salts that are not pharmaceutically acceptable when they are used as precursors to the free compounds or their pharmaceutically acceptable salts or in other synthetic manipulations.

It will be also understood that these alcohol compounds can be converted to the esters of phosphate, amino acid, acetic acid, etc, which can be used as pro-drugs to improve pharmacokinetic or pharmaceutical properties.

Solvates, and in particular, the hydrates of the compounds of structural formula I are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds of described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in viva half-life or reducing dosage requirements, or may provide a compound useful as a standard for characterization of biological samples. Isotopically-enriched compounds within generic formula can be prepared without undue experimentation by conventional techniques well known to those skilled in the art or by processes analogous to those described in the Schemes and Examples herein using appropriate isotopically-enriched reagents and/or intermediates.

### Methods of Treatment

Also encompassed by the present invention are methods of treating MCH-related diseases. The compounds described herein are effective in preventing or treating various MCH-related diseases, such as metabolic diseases such as obesity, diabetes, hormone secretion disorder, hyperlipemia, gout, fatty liver, and the like; circulatory diseases such as angina pectoris, acute/congestive cardiac insufficiency, myocardial infarction, coronary arteriosclerosis, hypertension, nephropathy, electrolyte abnormality, and the like; central and peripheral nervous system diseases such as bulimia, affective disorder, depression, anxiety, epilepsy, delirium, dementia, schizophrenia, attention deficit/hyperactivity disorder, dysmnesia, somnipathy, cognitive impairment, dyskinesia, dysesthesia, dysosmia, morphine resistance, drug dependence, alcohol dependence, and the like; reproductive system diseases such as infertility, premature delivery, sexual dysfunction, and the like; and other conditions including digestive diseases, respiratory diseases, cancer, and chromatosis. The compound of the invention is especially useful as a preventive or a remedy for obesity, diabetes, fatty liver, bulimia, depression, or anxiety.

One aspect of the invention described herein provides a method for the treatment and control of obesity or metabolic syndrome, which comprises administering to a patient in need of such treatment a therapeutically effective amount of a compound having the formulas described herein or a pharmaceutically acceptable salt thereof. For example, the compounds described herein are useful for treating or preventing obesity by administering to a subject in need thereof a composition comprising a compound of formula I, formula Ia or formula Ib.

Methods of treating or preventing obesity and conditions associated with obesity refer to the administration of the pharmaceutical formulations described herein to reduce or maintain the body weight of an obese subject or to reduce or maintain the body weight of an individual at risk of becoming obese. One outcome of treatment may be reducing the body weight of an obese subject relative to that subject's body weight immediately before the administration of the compounds or combinations of the present invention. Another outcome of treatment may be preventing body weight, regain of body weight previously lost as a result of diet, exercise, or pharmacotherapy and preventing weight gain from cessation of smoking. Another outcome of treatment may be decreasing the occurrence of and/or the severity of obesity-related diseases. Yet another outcome of treatment may be decreasing the risk of developing diabetes in an overweight or obese subject. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the pharmaceutical formulations described herein to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, type 2 diabetes, polycystic ovary disease, cardiovascular diseases, osteoarthritis, dermatological disorders, hypertension, insulin resistance, hypercholesterolemia, hypertriglyceridemia, and cholelithiasis.

Another aspect of the invention that is of interest relates to a method of treating hyperglycemia, diabetes or insulin resistance in a mammalian patient in need of such treatment which comprises administering to said patient a compound in accordance with the formulas described herein or a pharmaceutically acceptable salt thereof in an amount that is effective to treat hyperglycemia, diabetes or insulin resistance.

More particularly, another aspect of the invention that is of interest relates to a method of treating type 2 diabetes in a mammalian patient in need of such treatment comprising administering to the patient a compound in accordance with the formulas described herein or a pharmaceutically acceptable salt thereof in an amount that is effective to treat type 2 diabetes.

Yet another aspect of the invention that is of interest relates to a method of treating non-insulin dependent diabetes mellitus in a mammalian patient in need of such treatment comprising administering to the patient a compound in accordance with the formulas described herein or a pharmaceutically acceptable salt thereof in an amount that is effective to treat non-insulin dependent diabetes mellitus.

The present invention is also directed to the use of a compound of structural formula I, formula Ia or formula Ib in the manufacture of a medicament for use in treating various MCH-related diseases, such as metabolic diseases such as obesity, diabetes, hormone secretion disorder, hyperlipemia, gout, fatty liver, and the like; circulatory diseases such as angina pectoris, acute/congestive cardiac insufficiency, myocardial infarction, coronary arteriosclerosis, hypertension, nephropathy, electrolyte abnormality, and the like; central and peripheral nervous system diseases such as bulimia, affective disorder, depression, anxiety, epilepsy, delirium, dementia, schizophrenia, attention deficit/hyperactivity disorder, dysmnesia, somnipathy, cognitive impairment, dyskinesia, dysesthesia, dysosmia, morphine resistance, drug dependence, alcohol dependence, and the like; reproductive system diseases such as infertility, premature delivery, sexual dysfunction, and the like; and other conditions including digestive diseases, respiratory diseases, cancer, and chromatosis. The compounds described herein are especially useful as a preventive or a remedy for obesity, diabetes, fatty liver, bulimia, depression, or anxiety.

For example, the present invention is directed to the use of a compound of structural formula I, formula Ia or formula Ib in the manufacture of a medicament for use in treating obesity, diabetes, hormone secretion disorder, hyperlipemia, gout and fatty liver.

Additionally, the present invention is directed to the use of a compound of structural formula I in the manufacture of a medicament for use in treating obesity.

### Pharmaceutical Compositions

Compounds of the invention may be administered orally or parenterally. As formulated into a dosage form suitable for the administration route, the compound of the invention can be used as a pharmaceutical composition for the prevention, treatment, or remedy of the above diseases.

For clinical use of the compounds described herein, usually, the compound is formulated into various preparations together with pharmaceutically acceptable additives according to the dosage form, and may then be administered. By “pharmaceutically acceptable” it is meant the additive, carrier, diluent or excipient must be compatible with the other ingredients of the formulation and not deleterious to the recipient thereof. As such additives, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders, suppositories; and liquid preparations such as syrups, elixirs, injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9% by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. The dose is preferably from about 0.01 to about 25 mg/kg/day, more preferably from about 0.05 to about 10 mg/kg/day. For oral administration, the compositions are preferably provided in the form of tablets or capsules containing from 0.01 mg to 1,000 mg, preferably 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds of the formulas described herein or the other drugs may have utility, where the combination of the drugs together are safer or more effective than either drug alone. Such other drug(s) may be administered, by a route and in an amount commonly used therefore, contemporaneously or sequentially with a compound of formula I, Ia or Ib. When a compound of formula I, Ia or Ib is used contemporaneously with one or more other drugs, a pharmaceutical composition in unit dosage form containing such other drugs and the compound of formula I, Ia or Ib is preferred. However, the combination therapy may also include therapies in which the compound of formula I, Ia or Ib and one or more other drugs are administered on different overlapping schedules. It is also contemplated that when used in combination with one or more other active ingredients, the compounds described herein and the other active ingredients may be used in lower doses than when each is used singly. Accordingly, the pharmaceutical compositions described herein include those that contain one or more other active ingredients, in addition to a compound of formula I, Ia or Ib.

Examples of other active ingredients that may be administered in combination with a compound of formula I, Ia or Ib, and either administered separately or in the same pharmaceutical composition, include, but are not limited to:

(1) dipeptidyl peptidase-TV (DPP-4) inhibitors;

(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), such as those disclosed in WO 02/060388, WO 02/08188, WO 2004/019869, WO 2004/020409, WO 2004/020408, and WO 2004/066963, and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;

(3) insulin or insulin analogs, such as insulin lispro, insulin detemir, insulin glargine, insulin glulisine, and inhalable formulations of each thereof;

(4) leptin and leptin derivatives and agonists;

(5) amylin and amylin analogs, such as pramlintide;

(6) sulfonylurea and non-sulfonylurea insulin secretagogues, such as tolbutamide, glyburide, glipizide, glimepiride, mitiglinide, and meglitinides, such as nateglinide and repaglinide;

(7) α-glucosidase inhibitors (such as acarbose, voglibose and miglitol);

(8) glucagon receptor antagonists, such as those disclosed in WO 98/04528, WO 99/01423, WO 00/39088, and WO 00/69810;

(9) incretin mimetics, such as GLP-1, GLP-1 analogs, derivatives, and mimetics; and GLP-1 receptor agonists, such as exenatide, liraglutide, taspoglutide, AVE0010, CJC-1131, and BIM-51077, including intranasal, transdermal, and once-weekly formulations thereof;

(10) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;

(11) HDL-raising drugs, such as niacin or a salt thereof and extended-release versions thereof; MK-524A, which is a combination of niacin extended-release and the DP-1 antagonist MK-524; and nicotinic acid receptor agonists;

(12) antiobesity compounds;

(13) agents intended for use in inflammatory conditions, such as aspirin, non-steroidal anti-inflammatory drugs (NSAIDs), glucocorticoids, and selective cyclooxygenase-2 (COX-2) inhibitors;

(14) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;

(15) glucokinase activators (GKAs), such as LY2599506;

(16) inhibitors of 11β-hydroxysteroid dehydrogenase type 1, such as those disclosed in U.S. Pat. No. 6,730,690; WO 03/104207; and WO 04/058741;

(17) inhibitors of cholesteryl ester transfer protein (CETP), such as torcetrapib and MK-0859;

(18) inhibitors of fructose 1,6-bisphosphatase, such as those disclosed in U.S. Pat. Nos. 6,054,587; 6,110,903; 6,284,748; 6,399,782; and 6,489,476;

(19) inhibitors of acetyl CoA carboxylase-1 or 2 (ACC1 or ACC2);

(20) AMP-activated Protein Kinase (AMPK) activators;

(21) agonists of the G-protein-coupled receptors: GPR-109, GPR-119, and GPR-40;

(22) SSTR3 antagonists, such as those disclosed in WO 2009/011836;

(23) neuromedin U receptor agonists, such as those disclosed in WO2009/042053, including, but not limited to, neuromedin S (NMS);

(24) inhibitors of stearoyl-coenzyme A delta-9 desaturase (SCD);

(25) GPR-105 antagonists, such as those disclosed in WO 2009/000087;

(26) inhibitors of glucose uptake, such as sodium-glucose transporter (SGLT) inhibitors and its various isoforms, such as SGLT-1; SGLT-2, such as dapagliflozin and remogliflozin; and SGLT-3;

(27) inhibitors of acyl coenzyme A:diacylglycerol acyltransferase 1 and 2 (DGAT-1 and DGAT-2);

(28) inhibitors of fatty acid synthase;

(29) inhibitors of acetyl-CoA carboxylase-1 and 2 (ACC-1 and ACC-2);

(30) inhibitors of acyl coenzyme A:monoacylglycerol acyltransferase 1 and 2 (MGAT-1 and MGAT-2);

(31) agonists of the TGR5 receptor (also known as GPBAR1, BG37, GPCR19, GPR131, and M-BAR); and

(32) bromocriptine mesylate and rapid-release formulations thereof.

Dipeptidyl peptidase-IV (DPP-4) inhibitors that can be used in combination with compounds of formula I, Ia or Ib include, but are not limited to, sitagliptin (disclosed in U.S. Pat. No. 6,699,871), vildagliptin, saxagliptin, alogliptin, denagliptin, carmegliptin, dutogliptin, melogliptin, linagliptin, and pharmaceutically acceptable salts thereof, and fixed-dose combinations of these compounds with metformin hydrochloride, pioglitazone, rosiglitazone, simvastatin, atorvastatin, or a sulfonylurea.

Other dipeptidyl peptidase-IV (DPP-4) inhibitors that can be used in combination with compounds of formula I, Ia or Ib include, but are not limited to:
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-5-(1-methyl-4,6-dihydropyrrolo[3,4-c]pyrazol-5(1H)-yl)-2-(2,4,5-trifluorophenyl)tetrahydro-2H-pyran-3-amine;
(2R,3S,5R)-2-(2,5-difluorophenyl)tetrahydro)-5-(4,6-dihydropyrrolo[3,4-c]pyrazol-5(1H)-yl) tetrahydro-2H-pyran-3-amine;
(3R)-4-[(3R)-3-amino-4-(2,4,5-trifluorophenyl)butanoyl]-hexahydro-3-methyl-2H-1,4-diazepin-2-one;
4-[(3R)-3-amino-4-(2,5-difluorophenyl)butanoyl]hexahydro-1-methyl-2H-1,4-diazepin-2-one hydrochloride; and
(3R)-4-[(3R)-3-amino-4-(2,4,5-trifluorophenyl)butanoyl]-hexahydro-3-(2,2,2-trifluoroethyl)-2H-1,4-diazepin-2-one; and
   pharmaceutically acceptable salts thereof.

Antiobesity compounds that can be combined with compounds of formula I, Ia or Ib include topiramate; zonisamide; naltrexone; phentermine; bupropion; the combination of bupropion and naltrexone; the combination of bupropion and zonisamide; the combination of topiramate and phentermine; fenfluramine; dexfenfluramine; sibutramine; lipase inhibitors, such as orlistat and cetilistat; melanocortin receptor agonists, in particular, melanocortin-4 receptor agonists; CCK-1 agonists; melanin-concentrating hormone (MCH) receptor antagonists; neuropeptide Y₁ or Y₅ antagonists (such as MK-0557); CB1 receptor inverse agonists and antagonists (such as rimonabant and taranabant); β₃ adrenergic receptor agonists; ghrelin antagonists; bombesin receptor agonists (such as bombesin receptor subtype-3 agonists); and 5-hydroxytryptamine-2c (5-HT2c) agonists, such as lorcaserin. For a review of anti-obesity compounds that can be combined with compounds of the present invention, see S. Chaki et al., “Recent advances in feeding suppressing agents: potential therapeutic strategy for the treatment of obesity,” Expert Opin. Ther. Patents, 11: 1677-1692 (2001); D. Spanswick and K. Lee, “Emerging antiobesity drugs,” Expert Opin. Emerging Drugs, 8: 217-237 (2003); J. A. Fernandez-Lopez, et al., “Pharmacological Approaches for the Treatment of Obesity,” Drugs, 62: 915-944 (2002); and K. M. Gadde, et al., “Combination pharmaceutical therapies for obesity,” Exp. Opin. Pharmacother., 10: 921-925 (2009).

Glucagon receptor antagonists that can be used in combination with the compounds of formula I, Ia or Ib include, but are not limited to:
N-[4-((S)-1-{3-(3,5-dichlorophenyl)-5-[6-(trifluoromethoxy)-2-naphthyl]-1H-pyrazol-1-yl}ethyl)benzoyl]-β-alanine;
N-[4-((1R)-1-{3-(3,5-dichlorophenyl)-5-[6-(trifluoromethoxy)-2-naphthyl]-1H-pyrazol-1-yl}ethyl)benzoyl]-β-alanine;
N-(4-{1-[3-(2,5-dichlorophenyl)-5-(6-methoxy-2-naphthyl)-1H-pyrazol-1-yl]ethyl}benzoyl)-β-alanine;
N-(4-{(1S)-1-[3-(3,5-dichlorophenyl)-5-(6-methoxy-2-naphthyl)-1H-pyrazol-1-yl]ethyl}benzoyl)-β-alanine;
N-(4-{(1S)-1-[(R)-(4-chlorophenyl)(7-fluoro-5-methyl-1H-indol-3-yl)methyl]butyl}benzoyl)-β-alanine; and
N-(4-{(1S)-1-[(4-chlorophenyl)(6-chloro-8-methylquinolin-4-yl)methyl]butyl}benzoyl)-β-alanine; and
   pharmaceutically acceptable salts thereof.

Inhibitors of stearoyl-coenzyme A delta-9 desaturase (SCD) that can be used in combination with the compounds of formula I, Ia or Ib include, but are not limited to:
[5-(5-{4-[2-(trifluoromethyl)phenoxy]piperidin-1-yl}-1,3,4-thiadiazol-2-yl)-2H-tetrazol-2-yl]acetic acid;
(2′-{4-[2-(trifluoromethyl)phenoxy]piperidin-1-yl}-2,5′-bi-1,3-thiazol-4-yl)acetic acid;
(5-{3-[4-(2-bromo-5-fluorophenoxy)piperidin-1-yl]isoxazol-5-yl}-2H-tetrazol-2-yl)acetic acid;
(3-{3-[4-(2-bromo-5-fluorophenoxy)piperidin-1-yl]-1,2,4-oxadiazol-5-yl}-1H-pyrrol-1-yl)acetic acid;
(5-{5-[4-(2-bromo-5-fluorophenoxy)piperidin-1-yl]pyrazin-2-yl}-2H-tetrazol-2-yl)acetic acid; and
(5-{2-[4-(5-bromo-2-chlorophenoxy)piperidin-1-yl]pyrimidin-5-yl}-2H-tetrazol-2-yl)acetic acid;
   and pharmaceutically acceptable salts thereof.

Glucokinase activators that can be used in combination with the compounds of formula I, Ia or Ib include, but are not limited to:
3-(6-ethanesulfonylpyridin-3-yloxy)-5-(2-hydroxy-1-methyl-ethoxy)-N-(1-methyl-1H-pyrazol-3-yl)benzamide;
5-(2-hydroxy-1-methyl-ethoxy)-3-(6-methanesulfonylpyridin-3-yloxy)-N-(1-methyl-1H-pyrazol-3-yl)benzamide;
5-(1-hydroxymethyl-propoxy)-3-(6-methanesulfonylpyridin-3-yloxy)-N-(1-methyl-1H-pyrazol-3-yl)benzamide;
3-(6-methanesulfonylpyridin-3-yloxy)-5-(1-methoxymethyl-propoxy)-N-(1-methyl-1H-pyrazol-3-yl)benzamide;
5-isopropoxy-3-(6-methanesulfonylpyridin-3-yloxy)-N-(1-methyl-1H-pyrazol-3-yl)benzamide;
5-(2-fluoro-1-fluoromethyl-ethoxy)-3-(6-methanesulfonylpyridin-3-yloxy)-N-(1-methyl-1H-pyrazol-3-yl)benzamide;
3-({4-[2-(dimethylamino)ethoxy]phenyl}thio)-N-(3-methyl-1,2,4-thiadiazol-5-yl)-6-[(4-methyl-4H-1,2,4-triazol-3-yl)thio]pyridine-2-carboxamide;
3-({4-[(1-methylazetidin-3-yl)oxy]phenyl}thio)-N-(3-methyl-1,2,4-thiadiazol-5-yl)-6-[(4-methyl-4H-1,2,4-triazol-3-yl)thio]pyridine-2-carboxamide;
N-(3-methyl-1,2,4-thiadiazol-5-yl)-6-[(4-methyl-4H-1,2,4-triazol-3-yl)thio]-3-{[4-(2-pyrrolidin-1-ylethoxy)phenyl]thio}pyridine-2-carboxamide; and
3-[(4-{2-[(2R)-2-methylpyrrolidin-1-yl]ethoxy}phenyl)thio-N-(3-methyl-1,2,4-thiadiazol-5-yl)-6-[(4-methyl-4H-1,2,4-triazol-3-yl)thio]pyridine-2-carboxamide; and pharmaceutically acceptable salts thereof.

Agonists of the GPR-119 receptor that can be used in combination with the compounds of formula I, Ia or Ib include, but are not limited to:
rac-cis 5-chloro-2-{4-[2-(2-([5-(methylsulfonyl)pyridin-2-yl]oxy}ethyl)cyclopropyl]piperidin-1-yl)pyrimidine;
5-chloro-2-{4-[(1R,2S)-2-(2-[5-(methylsulfonyl)pyridin-2-yl]oxy}ethyl)cyclopropyl]piperidin-1-yl]pyrimidine;
rac cis-5-chloro-2-[4-(2-{2-[4-(methylsulfonyl)phenoxy]ethyl}cyclopropyl)piperidin-1-yl]pyrimidine;
5-chloro-2-[4-((1S,2R)-2-{2-[4-(methylsulfonyl)phenoxy]ethyl}cyclopropyl)piperidin-1-yl]pyrimidine;
5-chloro-2-[4-((1R,2S)-2-{2-[4-(methylsulfonyl)phenoxy]ethyl}cyclopropyl)piperidin-1-yl]pyrimidine;
rac cis-5-chloro-2-[4-(2-{2-[3-(methylsulfonyl)phenoxy]ethyl}cyclopropyl)piperidin-1-yl]pyrimidine; and
rac cis-5-chloro-2-[4-(2-{2-[3-(5-methyl-1,3,4-oxadiazol-2-yl)phenoxy]ethyl}cyclopropyl)piperidin-1-yl]pyrimidine; and pharmaceutically acceptable salts thereof.

Selective PPARγ modulators (SPPARγM's) that can be used in combination with the compounds of formula I, Ia or Ib include, but are not limited to:
(2S)-2-({6-chloro-3-[6-(4-chlorophenoxy)-2-propylpyridin-3-yl]-1,2-benzisoxazol-5-yl}oxy)propanoic acid;
(2S)-2-({6-chloro-3-[6-(4-fluorophenoxy)-2-propylpyridin-3-yl]-1,2-benzisoxazol-5-yl}oxy)propanoic acid;
(2S)-2-{[6-chloro-3-(6-phenoxy-2-propylpyridin-3-yl)-1,2-benzisoxazol-5-yl]oxy}propanoic acid;
(2R)-2-({6-chloro-3-[6-(4-chlorophenoxy)-2-propylpyridin-3-yl]-1,2-benzisoxazol-5-yl}oxy)propanoic acid;
(2R)-2-{3-[3-(4-methoxy)benzoyl-2-methyl-6-(trifluoromethoxy)-1H-indol-1-yl]phenoxy}butanoic acid;
(2S)-2-{3-[3-(4-methoxy)benzoyl-2-methyl-6-(trifluoromethoxy)-1H-indol-1-yl]phenoxy}butanoic acid;
2-{3-[3-(4-methoxy)benzoyl-2-methyl-6-(trifluoromethoxy)-1H-indol-1-yl]phenoxy}-2-methylpropanoic acid; and
(2R)-2-{3-[3-(4-chloro)benzoyl-2-methyl-6-(trifluoromethoxy)-1H-indol-1-yl]phenoxy}propanoic acid; and pharmaceutically acceptable salts thereof.

Inhibitors of 11β-hydroxysteroid dehydrogenase type 1 that can be used in combination with the compounds of formula I, Ia or Ib include, but are not limited to:
3-[1-(4-chlorophenyl)-trans-3-fluorocyclobutyl]-4,5-dicyclopropyl-r-4H-1,2,4-triazole; 3-[1-(4-chlorophenyl)-trans-3-fluorocyclobutyl]-4-cyclopropyl-5-(1-methylcyclopropyl)-r-4H-1,2,4-triazole;
3-[1-(4-chlorophenyl)-trans-3-fluorocyclobutyl]-4-methyl-5-[2-(trifluoromethoxy)phenyl]-r-4H-1,2,4-triazole;
3-[1-(4-chlorophenyl)cyclobutyl]-4-methyl-5-[2-(trifluoromethyl)phenyl]-4H-1,2,4-triazole;
3-{4-[3-(ethylsulfonyl)propyl]bicyclo[2.2.2]oct-1-yl}-4-methyl-5-[2-(trifluoromethyl)phenyl]-4H-1,2,4-triazole;
4-methyl-3-{4-[4-(methylsulfonyl)phenyl]bicyclo[2.2.2]oct-1-yl}-5-[2-(trifluoromethyl)phenyl]-4H-1,2,4-triazole;
3-(4-{4-methyl-5-[2-(trifluoromethyl)phenyl]-4H-1,2,4-triazol-3-yl}bicyclo[2.2.2]oct-1-yl)-5-(3,3,3-trifluoropropyl)-1,2,4-oxadiazole;
3-(4-{4-methyl-5-[2-(trifluoromethyl)phenyl]-4H-1,2,4-triazol-3-yl}bicyclo[2.2.2]oct-1-yl)-5-(3,3,3-trifluoroethyl)-1,2,4-oxadiazole;
5-(3,3-difluorocyclobutyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
5-(1-fluoro-1-methylethyl)-3-(4-{4-methyl-5-[2-(trifluoromethyl)phenyl]-4H-1,2,4-triazol-3-yl}bicyclo[2.2.2]oct-1-yl)-1,2,4-oxadiazole;
2-(1,1-difluoroethyl)-5-(4-{4-methyl-5-[2-(trifluoromethyl)phenyl]-4H-1,2,4-triazol-3-yl}bicyclo[2.2.2]oct-1-yl)-1,3,4-oxadiazole;
2-(3,3-difluorocyclobutyl)-5-(4-{4-methyl-5-[2-(trifluoromethyl)phenyl]-4H-1,2,4-triazol-3-yl}bicyclo[2.2.2]oct-1-yl)-1,3,4-oxadiazole; and
5-(1,1-difluoroethyl)-3-(4-{4-methyl-5-[2-(trifluoromethyl)phenyl]-4H-1,2,4-triazol-3-yl}bicyclo[2.2.2]oct-1-yl)-1,2,4-oxadiazole; and pharmaceutically acceptable salts thereof.

Somatostatin subtype receptor 3 (SSTR3) antagonists that can be used in combination with the compounds of formula I, Ia or Ib include, but are not limited to:

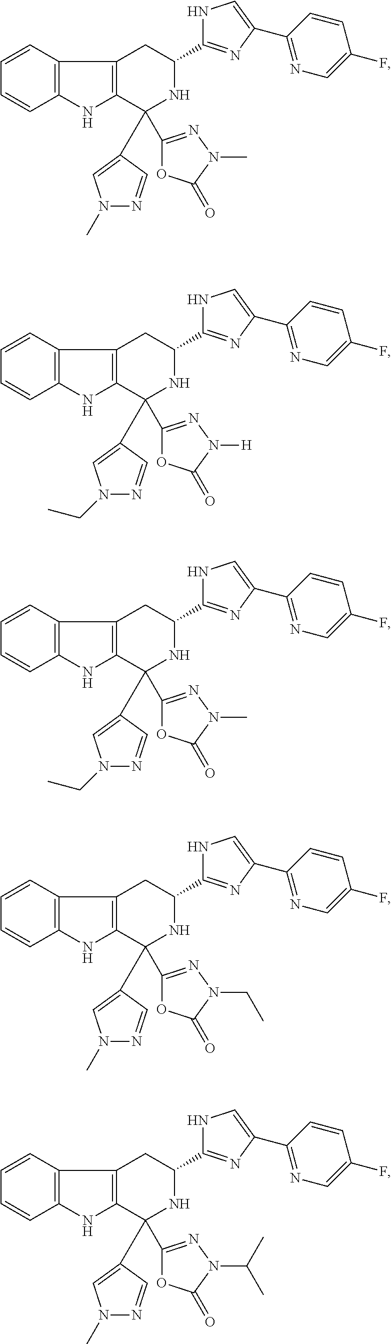

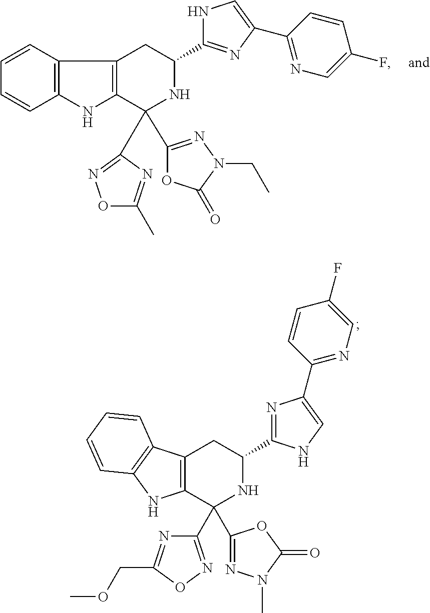

and pharmaceutically acceptable salts thereof.

AMP-activated Protein Kinase (AMPK) activators that can be used in combination with the compounds of formula I, Ia or Ib include, but are not limited to:

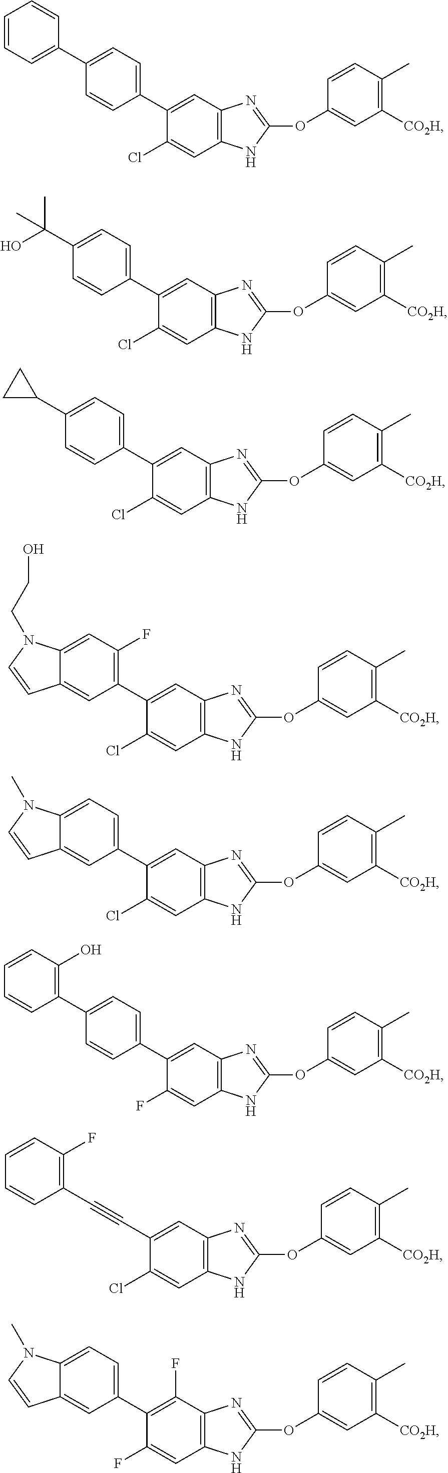

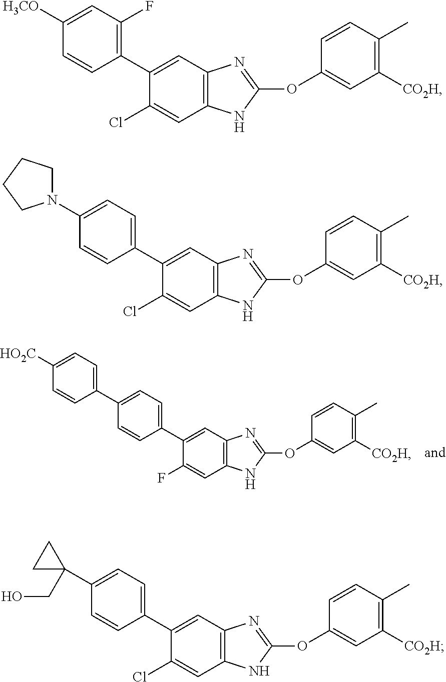

and pharmaceutically acceptable salts thereof.

Inhibitors of acetyl-CoA carboxylase-1 and 2 (ACC-1 and ACC-2) that can be used in combination with the compounds of formula I, Ia or Ib include, but are not limited to:
3-{1′-[(1-cyclopropyl-4-methoxy-1H-indol-6-yl)carbonyl]-4-oxospiro[chroman-2,4′-piperidin]-6-yl}benzoic acid;
5-{1′-[(1-cyclopropyl-4-methoxy-1H-indol-6-yl)carbonyl]-4-oxospiro[chroman-2,4′-piperidin]-6-yl}nicotinic acid;
1′-[(1-cyclopropyl-4-methoxy-1H-indol-6-yl)carbonyl]-6-(1H-tetrazol-5-yl)spiro[chroman-2,4′-piperidin]-4-one;
1′-[(1-cyclopropyl-4-ethoxy-3-methyl-1H-indol-6-yl)carbonyl]-6-(1H-tetrazol-5-yl)spiro[chroman-2,4′-piperidin]-4-one; and
5-{1′-[(1-cyclopropyl-4-methoxy-3-methyl-1H-indol-6-yl)carbonyl]-4-oxo-spiro[chroman-2,4′-piperidin]-6-yl}nicotinic acid; and
   pharmaceutically acceptable salts thereof.

In another aspect of the invention, a pharmaceutical composition is disclosed which comprises one or more of the following agents:

(a) a compound of structural formula I, formula Ia or formula Ib;

(b) one or more compounds selected from the group consisting of:
(1) dipeptidyl peptidase-IV (DPP-4) inhibitors;
(2) insulin sensitizers, including (i) PPARγ agonists, such as the glitazones (e.g. pioglitazone, rosiglitazone, netoglitazone, rivoglitazone, and balaglitazone) and other PPAR ligands, including (1) PPARα/γ, dual agonists, such as muraglitazar, aleglitazar, sodelglitazar, and naveglitazar, (2) PPARα agonists, such as fenofibric acid derivatives (gemfibrozil, clofibrate, ciprofibrate, fenofibrate and bezafibrate), (3) selective PPARγ modulators (SPPARγM's), and (4) PPARγ partial agonists; (ii) biguanides, such as metformin and its pharmaceutically acceptable salts, in particular, metformin hydrochloride, and extended-release formulations thereof, such as Glumetza®, Fortamet®, and GlucophageXR®; (iii) protein tyrosine phosphatase-1B (PTP-1B) inhibitors;
(3) sulfonylurea and non-sulfonylurea insulin secretagogues, such as tolbutamide, glyburide, glipizide, glimepiride, mitiglinide, and meglitinides, such as nateglinide and repaglinide;
(4) α-glucosidase inhibitors (such as acarbose, voglibose and miglitol);
(5) glucagon receptor antagonists;
(6) LDL cholesterol lowering agents such as (i) HMG-CoA reductase inhibitors (lovastatin, simvastatin, pravastatin, cerivastatin, fluvastatin, atorvastatin, pitavastatin, and rosuvastatin), (ii) bile acid sequestering agents (such as cholestyramine, colestimide, colesevelam hydrochloride, colestipol, and dialkylaminoalkyl derivatives of a cross-linked dextran, (iii) inhibitors of cholesterol absorption, such as ezetimibe, and (iv) acyl CoA:cholesterol acyltransferase inhibitors, such as avasimibe;
(7) HDL-raising drugs, such as niacin or a salt thereof and extended-release versions thereof; MK-524A, which is a combination of niacin extended-release and the DP-1 antagonist MK-524; and nicotinic acid receptor agonists;
(8) antiobesity compounds;
(9) agents intended for use in inflammatory conditions, such as aspirin, non-steroidal anti-inflammatory drugs (NSAIDs), glucocorticoids, and selective cyclooxygenase-2 (COX-2) inhibitors;
(10) antihypertensive agents, such as ACE inhibitors (such as enalapril, lisinopril, ramipril, captopril, quinapril, and tandolapril), A-II receptor blockers (such as losartan, candesartan, irbesartan, olmesartan medoxomil, valsartan, telmisartan, and eprosartan), renin inhibitors (such as aliskiren), beta blockers (such as and calcium channel blockers (such as;
(11) glucokinase activators (GKAs), such as LY2599506;
(12) inhibitors of 11β-hydroxysteroid dehydrogenase type 1;
(13) inhibitors of cholesteryl ester transfer protein (CETP), such as torcetrapib and MK-0859;
(14) inhibitors of fructose 1,6-bisphosphatase;
(15) inhibitors of acetyl CoA carboxylase-1 or 2 (ACC1 or ACC2);
(16) AMP-activated Protein Kinase (AMPK) activators;
(17) agonists of the G-protein-coupled receptors: GPR-109, GPR-119, and GPR-40;
(18) SSTR3 antagonists;
(19) neuromedin U receptor agonists, including, but not limited to, neuromedin S (NMS);
(20) inhibitors of stearoyl-coenzyme A delta-9 desaturase (SCD);
(21) GPR-105 antagonists;
(22) inhibitors of glucose uptake, such as sodium-glucose transporter (SGLT) inhibitors and its various isoforms, such as SGLT-1; SGLT-2, such as dapagliflozin and remogliflozin; and SGLT-3;
(23) inhibitors of acyl coenzyme A:diacylglycerol acyltransferase 1 and 2 (DGAT-1 and DGAT-2);
(24) inhibitors of fatty acid synthase;
(25) inhibitors of acetyl-CoA carboxylase-1 and 2 (ACC-1 and ACC-2);
(26) inhibitors of acyl coenzyme A:monoacylglycerol acyltransferase 1 and 2 (MCAT-1 and MGAT-2);
(27) agonists of the TGR5 receptor (also known as GPBAR1, BG37, GPCR19, GPR131, and M-BAR); and
(28) bromocriptine mesylate and rapid-release formulations thereof; and

(c) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

### EXAMPLES

Hereinafter, the invention will be described in further detail with reference to the following non-limiting examples.

The LC/MS analyses were preformed using a 6110 QUADRUPOLE mass spectrometer coupled to an AGILENT 1200 Series HPLC utilizing a ZORBAX SB-C18 2.1×50 mm column eluting at 0.8 mL/min with a solvent gradient of 1 to 90% B over 3.4 min, followed by a gradient of 90 to 100% B over 0.6 min, then followed by 0.5 min at 1% B: solvent A=0.04% TFA in water; solvent B=0.02% TFA in acetonitrile. ¹H-NMR spectra were obtained on a 400 MHz VARIAN Spectrometer in CDCl₃ or methanol-d4 as indicated and chemical shifts are reported as ppm using the solvent peak as reference and coupling constants are reported in hertz (Hz).

Abbreviations used in the following Schemes, Intermediate Examples, and Examples are: Ac₂O is acetic anhydride; b.p. is boiling point; DCM is dichloromethane; DMF is dimethylformamide; DMSO is dimethylsulfoxide; EtOAc is ethyl acetate; g is gram(s); h or hr is hours; HPLC is high performance liquid chromatography; IPA is isopropanol; LCMS or LC-MASS is liquid chromatography mass spectrum; M is molar; Me is methyl; MeCN is acetonitrile; MeOH is methanol; min is minutes; mg is milligram(s); mL is milliliter; mmHg is millimeter of mercury; mmol is millimole; MS or ms is mass spectrum; DIAD is diisopropyl azodicarboxylate; NMR is nuclear magnetic resonance; PE is petroleum ether; rt or RT is room temperature; aq is aqueous; t-BuOK is potassium tert-butoxide; THF is tetrahydrofuran; TLC is thin layer chromatography.

### Intermediate Example 1

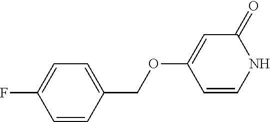

### 4-(4-Fluorobenzyloxy)pyridin-2(1H)-one

### Step A: 4-(4-Fluorobenzyloxy)pyridin-1-oxide

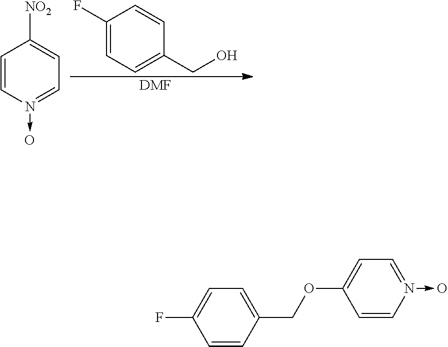

To a suspension of NaH (60%, 9.8 g, 410 mmol) in DMF (600 mL), 4-fluorobenzyl alcohol (57 g, 450 mmol) at 0° C. was added, and the resulting mixture was stirred at 25° C. for 1 h. Then, 4-nitropyridin-1-oxide (57 g, 410 mmol) was added in small batches, and the mixture was stirred for another 12 h. The volatiles were removed under reduced pressure, and the residue was treated with DCM (1000 mL). The crude product was collected by filtration, and was used in the next step without further purification.

### Step B: 4-(4-Fluorobenzyloxy)pyridin-2(1H)-one

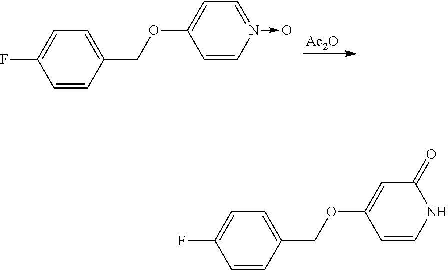

A sample of 4-(4-fluorobenzyloxy)pyridin-1-oxide (89 g, 410 mmol) in Ac₂O (700 mL) was heated at refluxed for 5 h. The volatiles were removed under reduced pressure, and the residue was dissolved in ethyl acetate (450 mL) and methanol (30 mL), which was heated at 60° C. for 3 h. The mixture was concentrated, and the residue was purified on a silica gel column eluting with PE:EtOAc (50:1) to EtOAc affording the title compound. ¹HNMR (400 MHz, CD₃OD, δ ppm): 5.05 (2H, s), 5.97 (1H, s), 6.13 (1H, d, J=7.2 Hz), 7.10 (2H, t, J=9.6 Hz), 7.30 (1H, d, J=8.0 Hz), 7.42-7.46 (2H, m). LCMS: m/e 200 [M+H]⁺.

### Intermediate Example 2

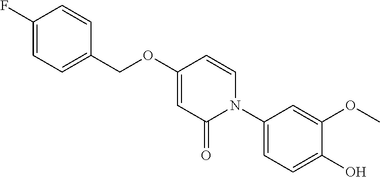

### 4-[(4-Fluorobenzyl)oxy]-1-(4-hydroxy-3-methoxyphenyl)pyridin-2(1H)-one

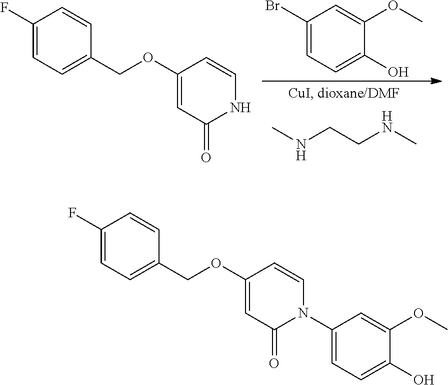

A mixture of 4-[(4-fluorobenzyl)oxy)pyridin-2(1H)-one (400 mg, 1.8 mmol), 4-bromo-2-methoxyphenol (480 mg, 2.4 mmol), CuI (350 mg, 1.8 mmol), N,N-dimethylethylenediamine (0.4 mL, 3.6 mmol) and K₃PO₄ (0.86 g, 3.6 mmol) and dioxane/DMF (10 mL/2 mL) was heated at 180° C. for 15 minutes in a microwave oven. The resulting mixture was diluted with DCM (30 mL), and was filtered. The filtrate was washed with aqueous ammonia (28%, 10 mL), and brine (10 mL), dried over Na₂SO₄, filtered and concentrated. The residue was purified on a silica gel column eluting with DCM:MeOH=98:2 to afford the title compound. ¹HNMR (400 MHz, CDCl₃, δ ppm): 3.82 (3H, s), 4.93 (2H, s), 5.93-5.97 (2H, m), 6.71 (1H, dd, J=8.4 Hz, 2.4 Hz), 6.81 (1H, d, J=2.4 Hz), 6.88 (1H, d, J=8.4 Hz), 7.03 (2H, t, J=8.4 Hz), 7.15 (1H, d, J=8.8 Hz), 7.31-7.34 (2H, m). LCMS: m/e 342 [M+H]⁺.

### Intermediate Example 3

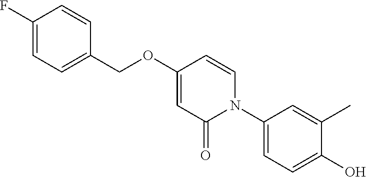

### 4-[(4-Fluorobenzyl)oxy]-1-(4-hydroxy-3-methylphenyl)pyridin-2(1H)-one

The title compound was prepared following the same procedure described for Intermediate Example 2 substituting 4-bromo-2-methoxyphenol with 4-bromo-2-methylphenol. ¹HNMR (400 MHz, CDCl₃, δ ppm): 2.07 (3H, s), 4.95 (2H, s), 5.99-6.02 (1H, m), 6.46 (1H, d, J=8.8 Hz), 6.76 (1H, dd, J=8.4 Hz, 2.4 Hz), 6.87 (1H, d, J=2.4 Hz), 7.04 (2H, t, J=8.4 Hz), 7.17 (1H, d, J=8.4 Hz), 7.35-7.31 (2H, m). LCMS: m/e 326 [M+H]⁺.

### Intermediate Example 4

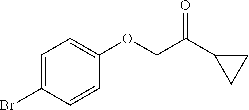

### 2-(4-Bromophenoxy)-1-cyclopropylethanone

### Step A: 2-Bromo-1-cyclopropyl-ethanone

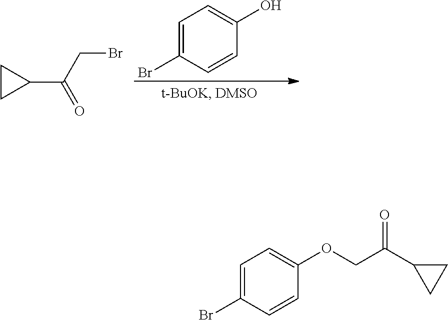

To a solution of 1-cyclopropylethanone (35 g, 42 mmol) in methanol (250 mL), bromine (21 mL, 420 mmol) was added while maintaining the reaction temperature below 20° C. After stirring at room temperature for 30 min, water (75 mL) was added, and stirring continued for 15 min. The resulting mixture was partitioned between water (200 mL) and ether (400 mL). The combined ether extracts were washed with saturated sodium carbonate and water, dried over magnesium sulfate, and filtered. The filtrated was distilled at reduced pressure to give the title compound.

### Step B: 2-(4-Bromophenoxy)-1-cyclopropylethanone

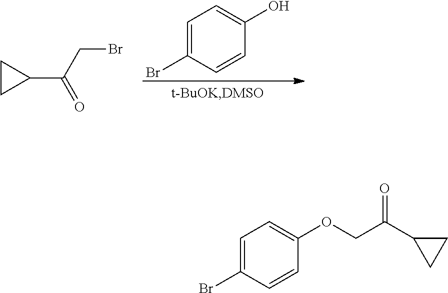

To a solution of 4-bromophenol (10 g, 58 mmol) in DMSO (100 mL) at rt, t-BuOK (6.5 g, 58 mmol) was added. After stirring for 10 min, 2-bromo-1-cyclopropylethanone (9.4 g, 58 mmol) was added, and stirring continued for 30 min. The resulting mixture was poured into cold aq K₂CO₃ solution (200 mL) and the product was extracted with ethyl acetate (3×300 mL). The combined organic layers were washed with brine, dried over Na₂SO₄, filtered, and concentrated, and the residue was purified on a silica gel column eluting with PE:EtOAc=10:1 to give the title compound.

### Intermediate Example 5

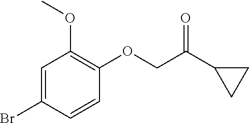

### 2-(4-Bromo-2-methoxy-phenoxy)-1-cyclopropylethanone

The title compound was prepared following the same procedure described for Intermediate Example 4 substituting 4-bromophenol with 4-bromo-2-methoxyphenol.

### Intermediate Example 6

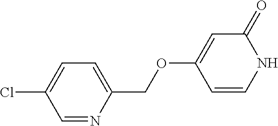

### 4-[(5-Chloropyridin-2-yl)methoxy]pyridin-2(1H)-one

The title compound was prepared following the same procedure described for Intermediate Example 1 substituting 4-fluorobenzyl alcohol with (5-chloropyridin-2-yl)methanol.

### Intermediate Example 7

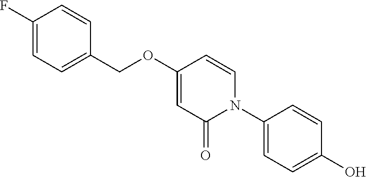

### 4-[(4-Fluorobenzyl)oxy]-1-(4-hydroxyphenyl)pyridin-2(1H)-one

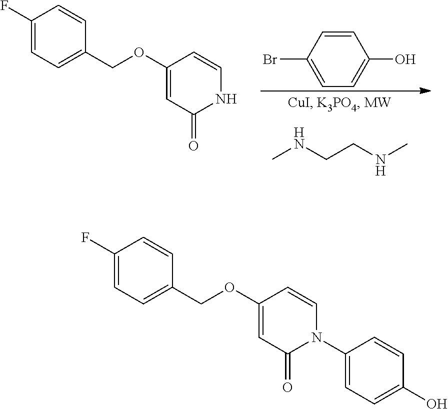

The title compound was prepared following the same procedure described for Reference Example 2 substituting 4-bromo-2-methoxyphenol with 4-bromophenol. ¹HNMR (400 MHz, CDCl₃, δ ppm): 7.37 (2H, t, 6.8 Hz), 7.19 (1H, s), 7.04 (2H, t, 8.8 Hz), 6.94 (2H, d, J=8.4 Hz), 6.97 (2H, d, J=9.2 Hz), 6.02 (2H, m), 4.95 (2H, s). LCMS: m/e 312 [M+H]⁺.

### Intermediate Example 8

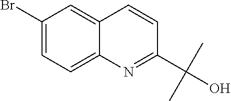

### Step A: 6-Bromoquinoline-2-carboxylic acid

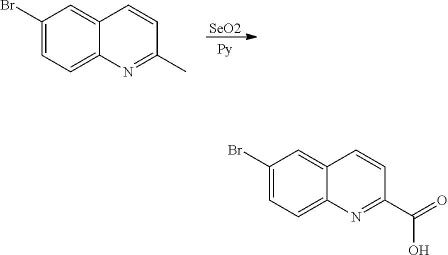

To a solution of 6-bromo-2-methylquinoline (100 mg, 0.45 mmol) in pyridine (5 mL), selenium dioxide (110 mg, 1.0 mmol) was added. After heating at 100° C. overnight, the resulting mixture was filtered, and the filtrate was concentrated. The residue was purified on a silica gel column eluting with PE:EtOAc (3:1 to 2:1) to afford the title compound. LCMS: m/e 252 [M+H]⁺.

### Step B: Methyl 6-bromoquinoline-2-carboxylate

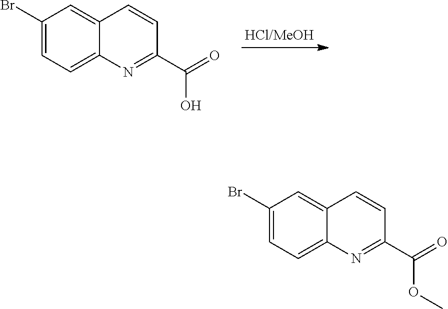

To a solution of 6-bromoquinoline-2-carboxylic acid (98 mg, 0.39 mmol) in MeOH (5 mL), hydrochloride gas was bubbled through for 5 min, and the mixture was heated at reflux for 5 h. Concentration of the resulting mixture afforded the title compound, which was used without further purification. LCMS: m/e 266 [M+H]⁺.

### Step C: 2-(6-Bromoquinolin-2-yl)propan-2-ol

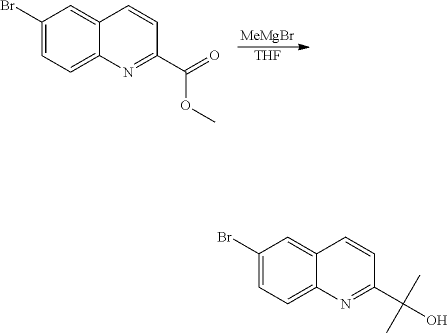

To a solution of methyl 6-bromoquinoline-2-carboxylate (100 mg, 0.39 mmol) in THF (1 mL) under N₂ at 0° C., methylmagnesium bromide ether (3.0 M, 0.26 mL, 0.78 mmol) was added dropwise. After stirring at room temperature for 2 h, the reaction was quenched with saturated aqueous ammonium chloride, and the product was extracted with ethyl acetate. The combined organic layers were washed with brine, dried over Na₂SO₄, filtered and concentrated to give the title compound. LCMS: m/e 266 [M+H]⁺.

### Example 1

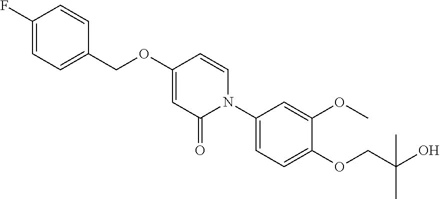

### 4-[(4-Fluorobenzyl)oxy]-1-[4-(2-hydroxy-2-methylpropoxy)-3-methoxyphenyl]pyridin-2(1H)-one

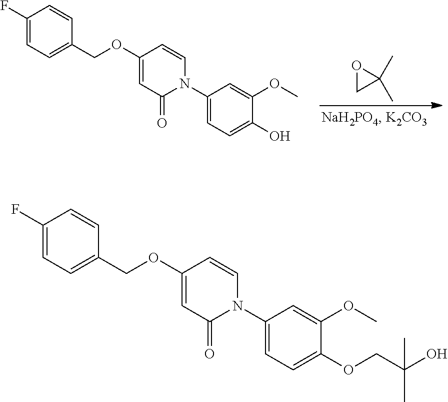

A mixture of 4-[(4-fluorobenzyl)oxy]-1-[(4-hydroxy-3-methoxyphenyl)]pyridine-2(1H)-one (50 mg, 0.15 mmol), NaH₂PO₄ hydrate (28 mg, 0.15 mmol), K₂CO₃ (40 mg, 0.29 mmol), 2,2-dimethyloxirane (18 mg, 025 mmol) and acetonitrile/water (1 mL/0.1 mL) was heated at 160° C. for 4 h in a microwave oven. The resulting mixture was acidified with TFA, and the product was obtained by preparative HPLC eluting with water/acetonitrile (containing 0.1% TFA, 37% to 67%). ¹HNMR (400 MHz, CD₃OD, δ ppm): 7.37-7.44 (3H, m), 7.04 (2H, t, J=8.8 Hz), 6.96 (1H, d, J=8.8 Hz), 6.89 (1H, d, J=2.4 Hz), 6.77 (1H, dd, J=8.8 Hz, 2.4 Hz), 6.16 (1H, dd, J=7.6 Hz, 2.8 Hz), 6.00 (1H, d, J=2.4 Hz), 5.04 (2H, s), 3.77 (3H, s), 3.73 (2H, s), 1.20 (6H, s). LCMS: m/e 414 [M+H]⁺

### Example 2

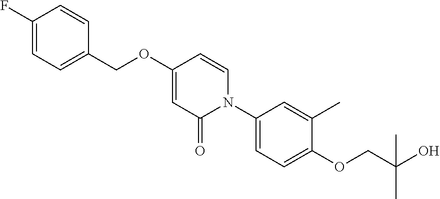

### 4-[(4-Fluorobenzyloxy)]-1-[4-(2-hydroxy-2-methylpropoxy)-3-methylphenyl]pyridin-2(1H)-one

The title compound was prepared following the same procedure as described for Example 1. ¹HNMR (400 MHz, CD₃OD, δ ppm): 7.43-7.46 (3H, m), 7.06-7.12 (4H, m), 6.94 (1H, d, J=8.0 Hz), 6.19 (1H, dd, J=7.6 Hz, 2.8 Hz), 6.04 (1H, d, J=2.4 Hz), 5.08 (2H, s), 3.79 (2H, s), 2.25 (3H, s), 1.31 (6H, s). LCMS: m/e 398 [M+H]⁺

### Example 3

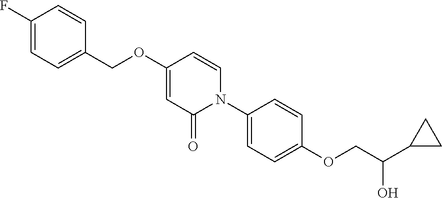

### 1-[4-(2-Cyclopropyl-2-hydroxyethoxy)phenyl]-4-[(4-fluorobenzyl)oxy]pyridin-2(1H)-one

### Step A: 1-[4-(2-Cyclopropyl-2-oxoethoxy)phenyl]-4-[4-fluorobenzyl)oxy]pyridin-2(1H)-one

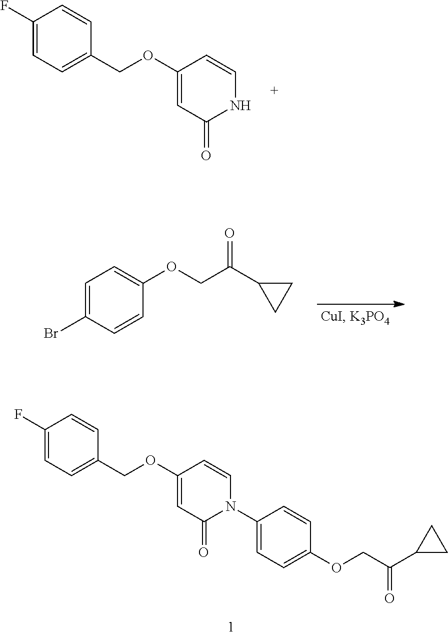

A mixture of 4-[(4-fluorobenzyl)oxy]pyridin-2(1H)-one (0.22 g, 1.0 mmol), 2-(4-bromophenoxy)-1-cyclopropylethanone (0.33 g, 1.3 mmol), CuI (0.19 g, 1.0 mmol), K₃PO₄ (0.42 g, 2.0 mmol) and N,N′-dimethylethylenediamine (0.18 g, 2.0 mmol) in dioxane (5 mL)/THF (1 mL) was heated at 160° C. for 20 min in a microwave oven. The resulting mixture was diluted with ethyl acetate (50 mL), washed with water (20 mL), brine, dried over Na₂SO₄, filtered and concentrated. The residue was purified on a silica gel column eluting with PE:acetone=1:1 to give the title compound. LCMS: m/e 394 [M+H]⁺.

### Step B: 1-[4-(2-Cyclopropyl-2-hydroxyethoxy)phenyl]-4-[4-fluorobenzyl)oxy]pyridin-2(1H)-one

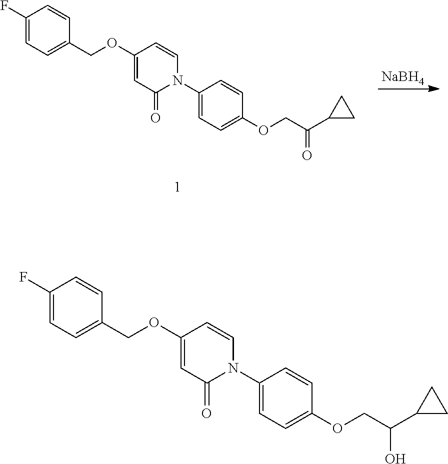

To a solution of 1-[4-(2-cyclopropyl-2-oxoethoxy)phenyl]-4-[(4-fluorobenzyl)oxy]pyridin-2(1H)-one (0.12 g, 0.31 mmol) in ethanol (3 mL) at 0° C., NaBH₄ (23 mg, 0.61 mmol) was added in portions. After stirring at rt for 1 h, the volatiles were removed under reduced pressure, and the residue was diluted with ethyl acetate (30 mL) and washed with water (10 mL), brine, dried over Na₂SO₄, filtered and concentrated to give the title compound in racemic form. ¹HNMR (400 MHz, CD₃OD, δ ppm): 0.34 (2H, m), 0.45 (2H, m), 0.92 (1H, m), 3.22 (1H, m), 3.93 (1H, m), 4.02 (1H, m), 5.03 (2H, s), 5.99 (1H, d, J=2.4 Hz), 6.15 (1H, dd, J=2.8 Hz, 7.6 Hz), 6.97-7.07 (4H, m), 7.17 (2H, d, J=8.8 Hz), 7.39 (3H, m). LCMS: Ink 396 [M+H]⁺. The title compound was further separated into its two enantiomers by preparative HPLC eluting on a Berger MultiGram™ SFC AD column (250×20 mm) eluting with 60% IPA in carbon dioxide. The retention time of enantiomer 1 is 5.49 min, and the retention time of enantiomer 2 is 6.63 min.

### Example 4

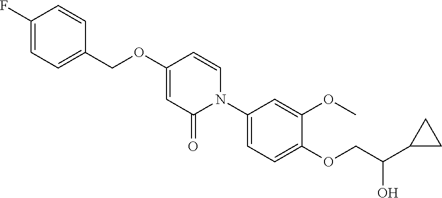

### 1-[4-(2-Cyclopropyl-2-hydroxyethoxy)-3-methoxyphenyl]-4-[(4-fluorobenzyl)oxy]pyridin-2(1H)-one

The title compound in racemic form was obtained following the same procedure as described for Example 3. ¹HNMR (400 MHz, CD₃OD, δ ppm): 0.33 (2H, m), 0.45 (2H, m), 0.91 (1H, m), 3.23 (1H, m), 3.76 (3H, s), 3.93 (1H, m), 4.03 (1H, m), 5.04 (2H, s), 5.99 (1H, d, J=2.8 Hz), 6.14 (1H, dd, J=2.4 Hz, 8.4 Hz), 6.77 (1H, dd, J=2.4 Hz, 8.4 Hz), 6.89 (1H, d, J=2.4 Hz), 6.98-7.07 (3H, m), 7.39 (3H, m). LCMS: m/e 426 [M+H]⁺. The title compound was further separated into its two enantiomers by preparative HPLC eluting on a Berger MultiGram™ SFC AD column (250×20 mm) eluting with 50% methanol in carbon dioxide. The retention time of enantiomer 1 is 4.66 min, and the retention time of enantiomer 2 is 5.94 min.

### Example 5

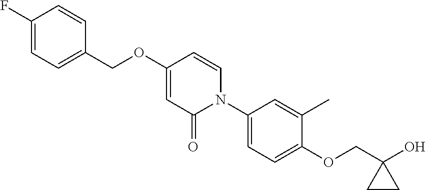

### 4-[(4-Fluorobenzyl)oxy]-1-{4-[(1-hydroxycyclopropyl)methoxy]-3-methylphenyl}pyridin-2(1H)-one

### Step A: Methyl (4-{4-[(4-fluorobenzyl)oxy]-2-oxopyridin-1(2H)-yl}-2-ethylphenoxy)acetate

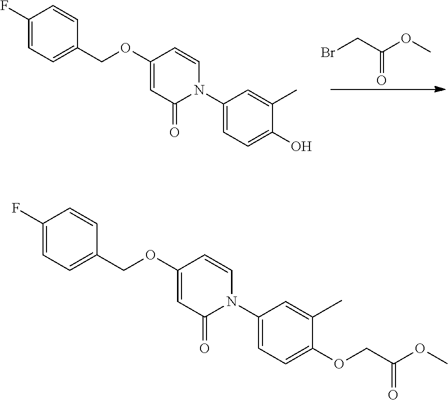

A mixture of 4-(4-fluorobenzyloxy)-1-(4-hydroxy-3-methylphenyl)pyridin-2(1H)-one (50 mg, 0.15 mmol), methyl 2-bromoacetate (100 mg, 0.77 mind), and K₂CO₃ (106 mg, 0.77 mmol) in MeCN (4 mL) was heated at 120° C. for 30 minutes in a microwave oven. The volatiles were removed under reduced pressure, and the residue was purified on a silica gel column eluting with DCM:MeOH 90:3 to afford the title compound. ¹HNMR (400 MHz, CD₃OD, δ ppm): 2.28 (3H, s), 176 (3H, s), 4.78 (2H, s), 5.09 (2H, s), 6.05 (1H, s), 6.19 (1H, dd, J=8.4 Hz, 2.4 Hz), 6.88 (1H, d, J=8.8 Hz), 7.06-7.14 (4H, m), 7.44-7.48 (3H, m). LCMS: m/e 398 [M+H]⁺.

### Step B: 4-(4-Fluorobenzyloxy)-1-{4-[(1-hydroxycyclopropyl)methoxy]-3-methylphenyl}pyridin-2(1H)-one

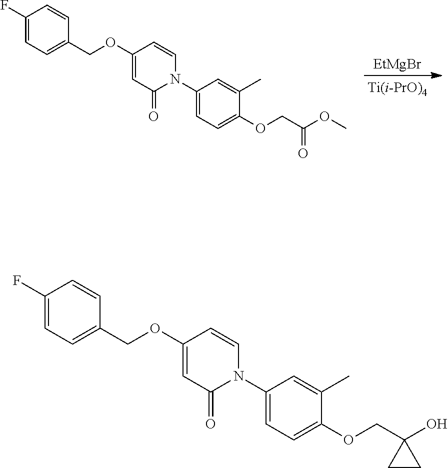

To a solution of methyl (4-{4-[(4-fluorobenzyl)oxy]-2-oxopyridin-1(2H)-yl}-2-ethylphenoxy)acetate (40 mg, 0.1 mmol) and tetraisopropoxytitanium (28 mg, 0.1 mmol) in anhydrous THF (3 mL) at 15° C. under nitrogen was added dropwise EtMgBr (0.09 mL, 0.27 mmol, 3M in Et₂O) pre-diluted with 1 mL of anhydrous THF. After stirring at rt for 1 h, the reaction was quenched with saturated NH₄Cl (5 mL), and the product was extracted with DCM (50 mL). The organic layer was separated and concentrated, and the residue was purified by preparative HPLC eluting with water/acetonitrile (containing 0.1% TFA, 30% to 70%) affording the title compound. ¹HNMR (400 MHz, CD₃OD, δ ppm): 0.78-0.80 (2H, m), 0.84-0.86 (2H, m), 2.31 (3H, s), 4.09 (2H, s), 5.15 (2H, s), 6.10 (1H, s), 6.26 (1H, dd, J=7.6 Hz, 2.4 Hz), 7.00 (1H, d, J=8.4 Hz), 7.10-7.18 (4H, m), 7.49-7.53 (3H, m). LCMS: m/e 396 [M+H]⁺.

### Example 6

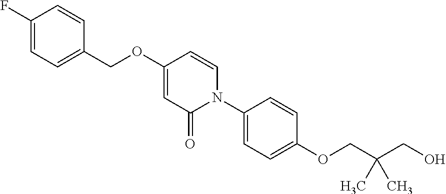

### 4-[(4-Fluorobenzyl)oxy]-1-[4-(3-hydroxy-2,2-dimethylpropoxy)phenyl]pyridin-2(1H)-one

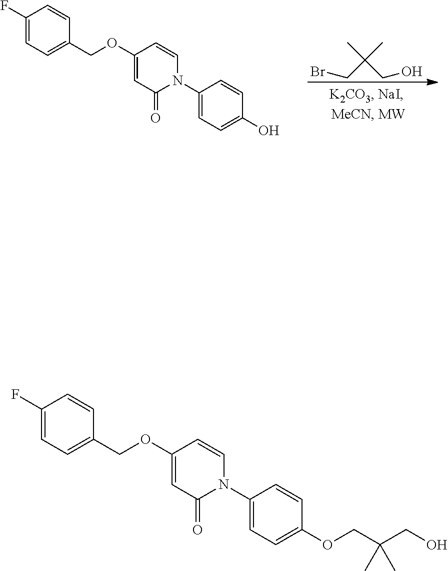

A mixture of 4-[(4-fluorobenzyl)oxy]-1-(4-hydroxyphenyl)pyridin-2(1H)-one (50 mg, 0.16 mmol), K₂CO₃ (110 mg, 0.800 mmol), 3-bromo-2,2-dimethyl-1-propanol (84 mg, 0.80 mmol) and NaI (12 mg, 0.08 mmol) in CH₃CN (4 mL) was heated at 160° C. for 6 h in a microwave oven. The volatiles were removed, and the residue was purified with preparative HPLC eluting with water/acetonitrile (containing 0.1% TFA, 30% to 70%) to afford the title compound. ¹NMR (400 MHz, CD₃OD, δ ppm): 7.37-7.41 (3H, m), 7.15 (2H, d, J=8.8 Hz), 7.04 (2H, t, J=8.8 Hz), 6.94 (2H, d, J=8.8 Hz), 6.13-6.16 (1H, m), 5.99 (1H, d, J=2.8 Hz), 5.03 (2H, s), 3.69 (2H, s), 3.37 (2H, s), 0.92 (6H, s). LCMS: m/e 398 [M+H]⁺.

### Example 7

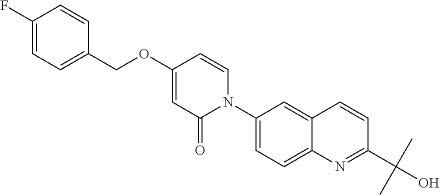

### 4-(4-Fluorobenzyloxy)-1-[2-(2-hydroxypropan-2-yl)quinolin-6-yl]pyridin-2-(1H)-one

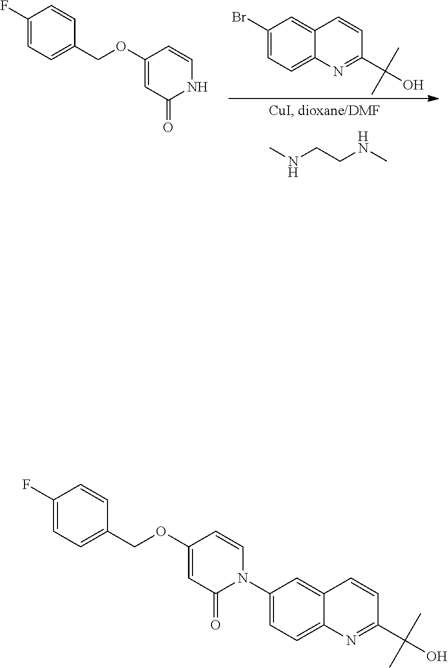

The title compound was prepared following the same procedure as described for Example 3. ¹HNMR (400 MHz, CD₃OD, δ ppm): 8.81-8.87 (1H, d, J=8.4 Hz), 8.39-8.46 (1H, d, J=9.2 Hz), 8.20 (1H, s), 8.05-8.09 (1H, d, J=8.4 Hz), 7.97-8.05 (1H, d, J=7.2 Hz), 7.66-7.68 (1H, d, J=7.6 Hz), 7.43-7.53 (2H, m), 7.13 (2H, t, J=8.8 Hz), 6.28-6.37 (1H, d, 8.0 Hz), 6.13 (1H, s), 5.15 (2H, s), 1.72 (611, LCMS: m/e 405 [M+H]⁺

### Example 8

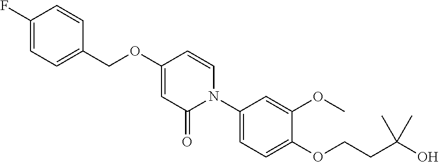

### 4-(4-Fluorobenzyloxy)-1-[4-(3-hydroxy-3-methylbutoxy)-3-methoxyphenyl]pyridin-2-(1H)-one

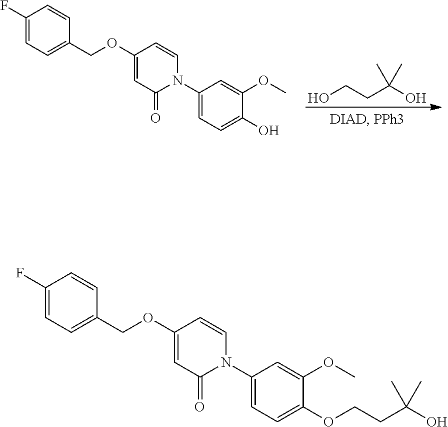

To a solution of 4-[(4-fluorobenzyloxy)-1-(4-hydroxy-3-methoxyphenyl)pyridin-2(1H)-one (25 mg, 0.073 mmol), 3-methylbutane-1,3-diol (11 mg, 0.11 mmol) and triphenylphosphine (19 mg, 0.073 mmol) in anhydrous THF (0.5 mL) under N₂ atmosphere at 0° C., diisopropyl azodicarboxylate (14 mg, 0.080 mmol) was added. After stirring at rt overnight, the volatiles were removed under reduced pressure, and the residue was purified with preparative HPLC eluting with water/acetonitrile (containing 0.1% TFA, 37% to 67%) to afford the title compound. ¹HNMR (400 MHz, CDCl₃, δ ppm): 7.39-7.53 (3H, m), 7.12 (2H, t, J=8.8 Hz), 7.04-7.09 (1H, m), 6.88-6.97 (1H, m), 6.78-6.86 (1H, m), 6.20-6.28 (1H, m), 6.05-6.09 (1H, m), 5.12 (2H, s), 4.12-4.21 (2H, m), 3.84 (3H, s), 1.99 (2H, t, J=6.8 Hz), 1.26 (6H, s). LCMS: m/e 428 [M+H]⁺.

### Example 9

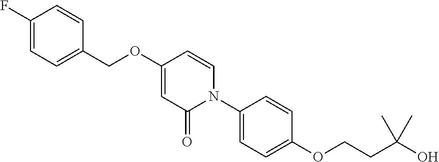

### 4-[(4-Fluorobenzyl)oxy]-1-[4-(3-hydroxy-3-methylbutoxy)phenyl]pyridin-2 (1H)-one

### Step A: 3-Hydroxy-3-methylbutyl 4-methylbenzenesulfonate

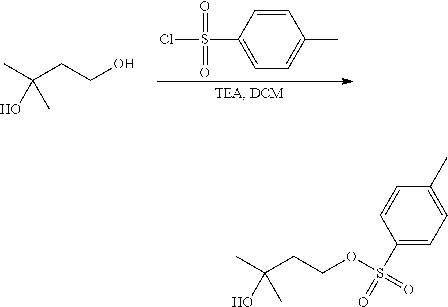

To a solution of 3-methylbutane-1,3-diol (1.0 g, 9.6 mmol) and triethylamine (1.6 mL, 11 mmol) in 10 mL of methylene chloride at 0° C., 4-methylbenzenesulfonyl chloride (2.0 g, 4.4 mmol) in 5 mL of methylene chloride was added slowly. After stirring at 0° C. for 30 min, the reaction was quenched with the addition of water (10 mL), and the pH of the aq layer was adjusted to 10 with 1M NaOH. The organic layer was separated, washed with water (2×), dried over magnesium sulfate, filtered and concentrated to give title compound, which was used without further purification. ¹HNMR (400 MHz, DMSO_d₆, δ ppm): 7.54 (2H, d, J=8.4 Hz), 7.52 (2H, d, J=8.0 Hz), 4.36 (1H, s), 4.07 (2H, t, J=7.2 Hz), 2.39 (3H, s), 1.66 (2H, s), 1.0 (6H, s).

### Step B: 4-[(4-Fluorobenzyl)oxy]-1-[4-(3-hydroxy-3-methylbutoxy)phenyl]pyridin-2(1H)-one

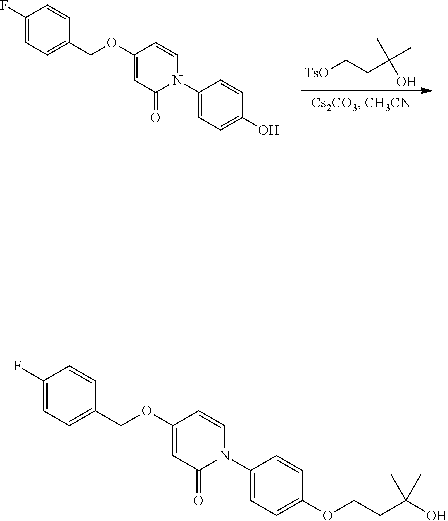

A mixture of 4-[(4-fluorobenzyl)oxy]-1-(4-hydroxyphenyl)pyridin-2(1H)-one (50 mg, 0.1608 mmol), Cs₂CO₃ (260 mg, 0.804 mmol), NaI (12 mg, 0.08 mmol) in CH₃CN (4 mL) was heated at 180° C. for 15 minutes in a microwave oven. The volatiles were removed under reduced pressure, and the residue was purified with preparative HPLC eluting with water/acetonitrile (containing 0.1% TFA, 30% to 70%) to afford the title compound. ¹HNMR (400 MHz, CD₃OD, δ ppm): 7.45-7.49 (3H, m), 7.23 (2H, d, J=6.8 Hz), 7.12 (2H, m), 7.01 (2H, d, J=8.8 Hz), 6.22 (1H, d, J=7.6 Hz), 6.07 (1H, d, J=2.4 Hz), 5.11 (2H, s), 4.16 (2H, t, J=6.0 Hz), 1.96 (2H, t, J=6.8 Hz), 1.26 (6H, s). LCMS: m/e 398 [M+H]⁺.

The following examples shown in Table 2, were made following the appropriate procedure described for Examples 1-9.

TABLE 2

| | | | LC-MS |
|---|---|---|---|
| Ex | Name | Structure | (m/e) |
| 10 | 1-[3-chloro-4-(2-hydroxy-2- methylpropoxy)phenyl]-4-[(4- fluorobenzyl)oxy]pyridin-2(1H)-one | 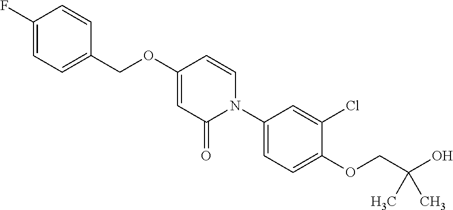 | 418 |
| 11 | 4-[(4-fluorobenzyl)oxy]-1-[4-(2-hydroxy-2- methylpropoxy)phenyl]pyridin-2(1H)-one | 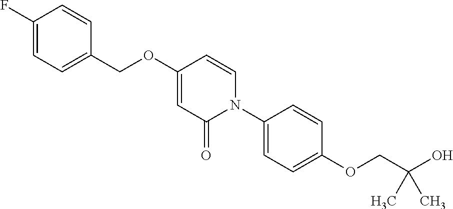 | 384 |
| 12 | 4-[(4-fluorobenzyl)oxy]-1-[3-fluoro-4-(2- hydroxy-2-methylpropoxy)phenyl]pyridin- 2(1H)-one | 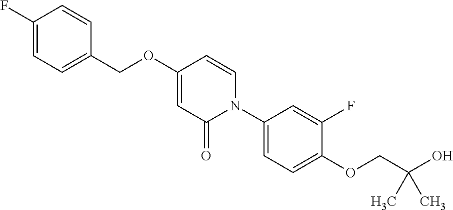 | 402 |
| 13 | 4-[(4-fluorobenzyl)oxy]-1-[4-(3-hydroxy-2,2- dimethylpropoxy)-3-methoxyphenyl]pyridin- 2(1H)-one | 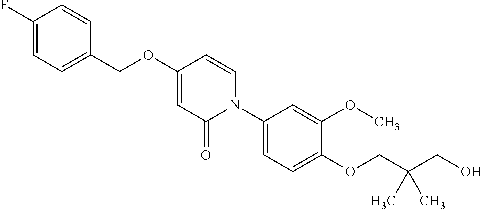 | 428 |
| 14 | 4-[(5-chloropyridin-2-yl)methoxy]-1-[4-(2- cyclopropyl-2-hydroxyethoxy)-3- methoxyphenyl]pyridin-2(1H)-one | 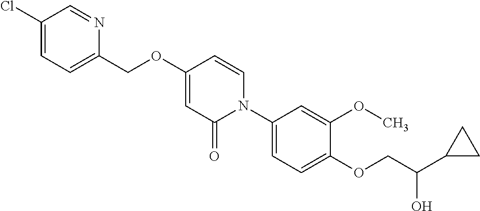 | 443 |
| 15 | 4-[(5-chloropyridin-2-yl)methoxy]-1-[4-(2- cyclopropyl-2-hydroxyethoxy)phenyl]pyridin- 2(1H)-one | 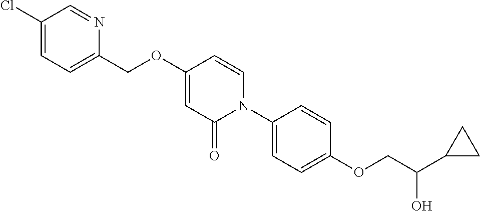 | 413 |
| 16 | 1-[4-(2-cyclopropyl-2- hydroxyethoxy)phenyl]-4-[(3,4- difluorobenzyl)oxy]pyridin-2(1H)-one | 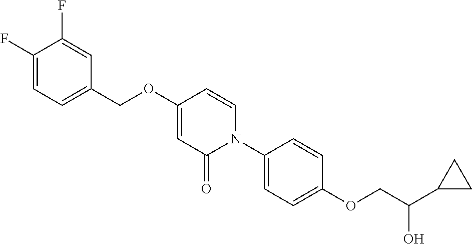 | 414 |
| 17 | methyl (2-fluoro-4-{4-[(4-fluorobenzyl)oxy]- 2-oxopyridin-1(2H)-yl}phenoxy)acetate | 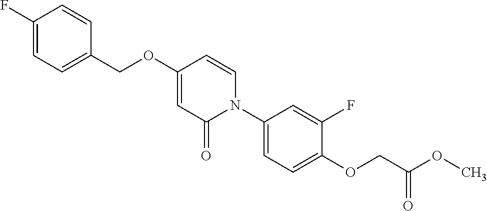 | 402 |
| 18 | propan-2-yl (4-{4-[(4-fluorobenzyl)oxy]-2- oxopyridin-1(2H)-yl}-2- methylphenoxy)acetate | 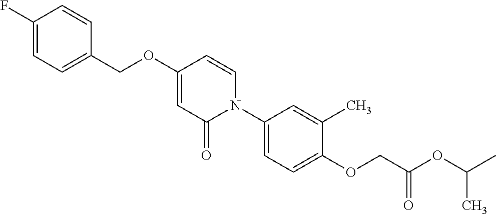 | 426 |
| 19 | methyl (4-{4-[(4-fluorobenzyl)oxy]-2- oxopyridin-1(2H)-yl}-2- methylphenoxy)acetate | 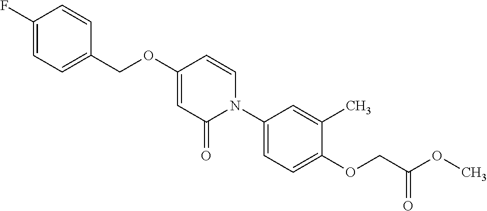 | 398 |
| 20 | methyl (4-{4-[(4-fluorobenzyl)oxy]-2- oxopyridin-1(2H)-yl}-2- methoxyphenoxy)acetate | 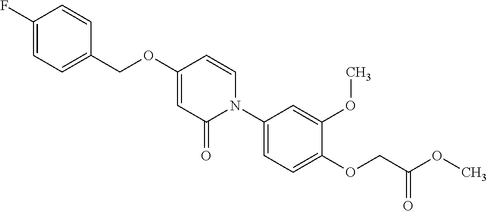 | 414 |
| 21 | 4-[(5-chloropyridin-2-yl)methoxy]-1-{4- [(2R)-2-cyclopropyl-2-hydroxyethoxy]-3- methoxyphenyl}pyridin-2(1H)-one | 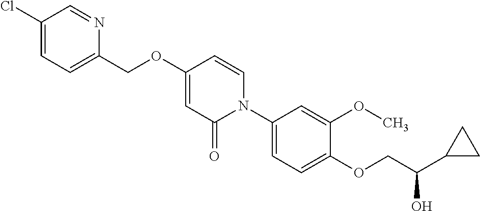 | 443 |
| 22 | 4-[(5-chloropyridin-2-yl)methoxy[-1-{4- [(2R)-2-cyclopropyl-2- hydroxyethoxy]phenyl}pyridin-2(1H)-one | 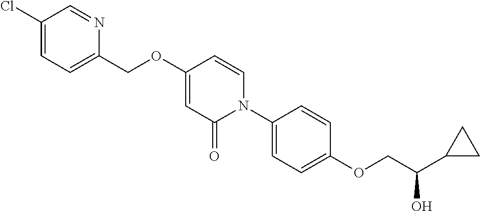 | 413 |
| 23 | 1-{4-[(2R)-2-cyclopropyl-2- hydroxyethoxy]phenyl}-4-[(3,4- difluorobenzyl)oxy]pyridin-2(1H)-one | 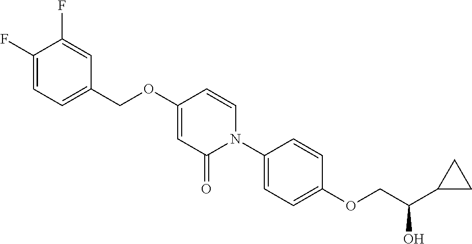 | 414 |
| 24 | 4-[(5-chloropyridin-2-yl)methoxy]-1-{4- [(2S)-2-cyclopropyl-2-hydroxyethoxy]-3- methoxyphenyl}pyridin-2(1H)-one | 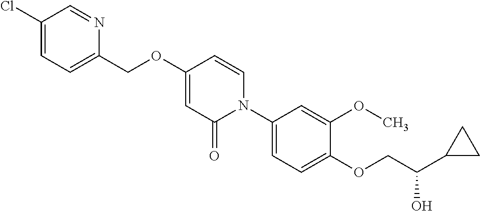 | 443 |
| 25 | 1-{4-[(2S)-2-cyclopropyl-2- hydroxyethoxy]phenyl}-4-[(3,4- difluorobenzyl)oxy]pyridin-2(1H)-one | 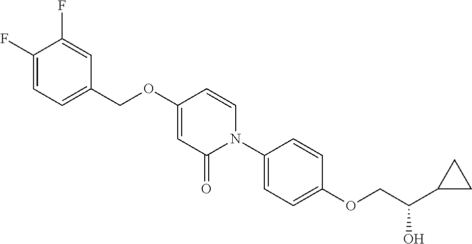 | 414 |
| 26 | 4-[(5-chloropyridin-2-yl)methoxy]-1-{4- [(2S)-2-cyclopropyl-2- hydroxyethoxy]phenyl}pyridin-2(1H)-one | 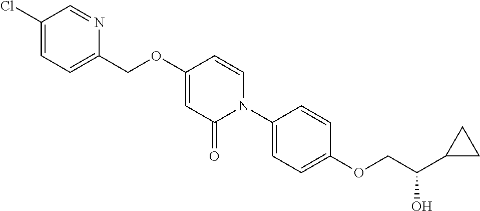 | 413 |
| 27 | 1-{3-chloro-4-[(1- hydroxycyclopropyl)methoxy]phenyl}-4-[(4- fluorobenzyl)oxy]pyridin-2(1H)-one | 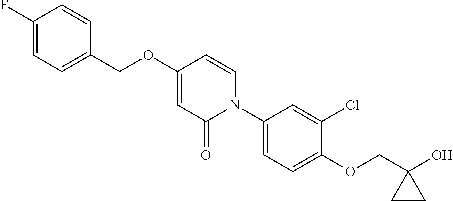 | 416 |
| 28 | 4-[(4-fluorobenzyl)oxy]-1-{3-fluoro-4-[(1- hydroxycyclopropyl)methoxy]phenyl}pyridin- 2(1H)-one | 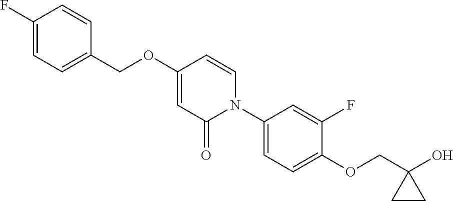 | 400 |
| 29 | 4-[(4-fluorobenzyl)oxy]-1-{4-[(1- hydroxycyclopropyl)methoxy]-3- methoxyphenyl}pyridin-2(1H)-one | 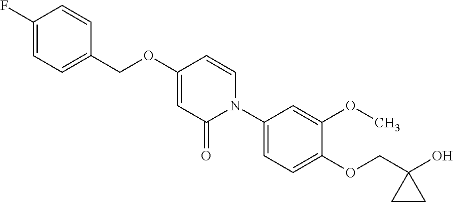 | 412 |
| 30 | 1-{4-[(2S)-2-cyclopropyl-2-hydroxyethoxy]- 3-methoxyphenyl}-4-[(3,4- difluorobenzyl)oxy]pyridin-2(1H)-one | 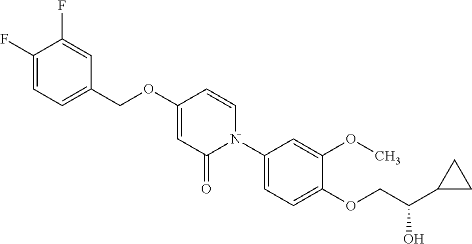 | 444 |
| 31 | 1-{4-[(2R)-2-cyclopropyl-2-hydroxyethoxy]- 3-methoxyphenyl}-4-[(3,4- difluorobenzyl)oxy]pyridin-2(1H)-one | 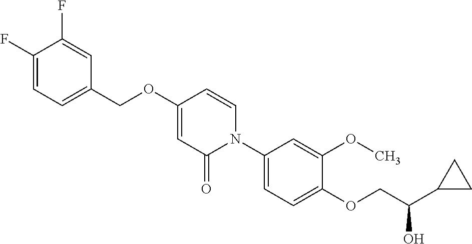 | 444 |

### MCH-1R Binding Inhibition:

A cDNA sequence encoding human MCH-1R [FEBS Letters, Vol. 398, 253 (1996); Biochimica et Biophisica Acta, Vol. 1401, 216 (1998)] was cloned to a plasmid vector pEF/myc/cyto (manufactured by Invitrogen). The obtained expression vector was transfected to a host cell CHO-K1 (American Type Culture Collection) using Lipofectamine Plus reagent (manufactured by Life Technology) to provide MCH-1R expression cells.

Membrane samples prepared from the MCH-1R expression cells were incubated with a test compound and 50 pM [¹²⁵I]MCH (manufactured by NEN) in an assay buffer (50 mM Tris buffer containing 10 mM magnesium chloride, 2 mM ethylenediamine tetraacetate, 0.01% bacitracin, and 0.2% bovine serum albumin; pH 7.4) at 25° C. for one hour, followed by filtration through a glass filter GF/C (manufactured by Wattman). The glass filter was washed with 50 mM Tris buffer (pH 7.4) containing 10 mM magnesium chloride, 2 mM ethylenediamine tetraacetate, and 0.04% Tween-20, and then the radioactivity on the glass filter was determined. Non-specific binding was measured in the presence of 1 μM human MCH, and, with respect to each test compound, 50% inhibition concentration (IC₅₀ value) for specific [¹²⁵I]MCH binding was determined. Examples 1-31 had an IC₅₀ value of less that 500 nM. Specific IC₅₀ values are shown in Table 3:

| Example | IC50 (nM) |
|---|---|
| 2 | 11 |
| 5 | 11 |
| 6 | 63 |
| 7 | 38 |
| 8 | 84 |
| 9 | 23 |
| 10 | 11 |
| 12 | 97 |
| 18 | 23 |
| 19 | 31 |
| 20 | 61 |
| 21 | 33 |
| 22 | 101 |
| 24 | 36 |
| 26 | 91 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of formula (I): 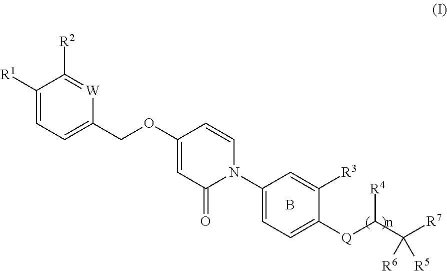
or a pharmaceutically acceptable salts thereof, wherein R¹ and R² are independently selected from the group consisting of halogen, hydrogen, —OH, C₁-C₆alkyl, —OC₁-C₆alkyl, —O-halogen-substitutedC₁-C₆ alkyl and halogen-substitutedC₁-C₆ alkyl;
W is —N— or —CH—;
Q is —O—, —NH—, or —C—, or taken together with R⁴, aromatic ring B and R³ form a heteroaryl;
R³ is halogen, hydrogen, —OC₁-C₆alkyl, C₁-C₆alkyl, —O-halogen-substitutedC₁-C₆alkyl, halogen-substitutedC₁-C₆alkyl, cyano, SO₂C₁-C₆alkyl or when taken together with aromatic ring B, Q and R⁴ form a heteroaryl ring;
R⁴ is hydrogen, oxo, C₁-C₆alkyl, halogen-substitutedC₁-C₆alkyl or together with aromatic ring B, R³ and Q form a heteroaryl or when taken together with R⁵ form a C₃-C₆cycloalkyl;
R⁵, R⁶ and R⁷ are each independently selected from the group consisting of hydrogen, C₁-C₆alkyl, halogen-substitutedC₁-C₆alkyl, C₃-C₆cycloalkyl, halogen-substitutedC₃-C₆cycloalkyl, C₁-C₆alkylC₃-C₆cycloalkyl, —OH, C₁-C₆alkyl-OH and —OC₁-C₆alkyl, or when R⁵ and R⁶ are taken together form an oxo group or C₃-C₆cycloalkyl, or when R⁵ and R⁴ are taken together form a C₃-C₆cycloalkyl, wherein at least one of R⁵, R⁶ and R⁷ is not hydrogen;
n is 1-3.

2. The formula of claim 1, or a pharmaceutically acceptable salt thereof, wherein R¹ and R² are halogen, and wherein the halogen is selected from the group consisting of fluorine and chlorine.

3. The formula of claim 1, or a pharmaceutically acceptable salt thereof, wherein R¹ is halogen, and wherein the halogen is selected from the group consisting of fluorine, and chlorine and wherein R² is hydrogen or methyl.

4. The formula of claim 1, or a pharmaceutically acceptable salt thereof, wherein W is —N—.

5. The formula of claim 1, or a pharmaceutically acceptable salt thereof, wherein W is —CH—.

6. The formula of claim 1, or a pharmaceutically acceptable salt thereof, wherein Q is —O—.

7. The formula of claim 1, or a pharmaceutically acceptable salt thereof, wherein R³ is hydrogen or —OC₁-C₆alkyl.

8. The formula of claim 1, or a pharmaceutically acceptable salt thereof, wherein R³ is —OC₁-C₆alkyl.

9. The formula of claim 1, or a pharmaceutically acceptable salt thereof, wherein R⁵, R⁶, R⁷ are independently selected from the group consisting of hydrogen, C₁-C₆alkyl-OH and —OH or when taken together two of R⁵, R⁶ and R⁷ are cyclopropyl.

10. The formula of claim 1, or a pharmaceutically acceptable salt thereof, wherein R⁵, R⁶, R⁷ are independently selected from the group consisting of hydrogen, —OH, C₁-C₆alkyl-OH and C₁-C₆alkylC₃-C₆cycloalkyl.

11. The formula of claim 1, or a pharmaceutically acceptable salt thereof, wherein R⁵ and R⁶ together form an oxo group and R⁷ is —OC₁-C₆alkyl.

12. The formula of claim 1, or a pharmaceutically acceptable salt thereof, wherein R⁵ and R⁶ together are cyclopropyl and R⁷ is —OC₁-C₆alkyl.

13. The formula of claim 1, and pharmaceutically acceptable salts thereof, wherein n is 1.

14. The formula of claim 1, or a pharmaceutically acceptable salt thereof, wherein n is 2.

15. A compound of claim 1 comprising formula (Ia): 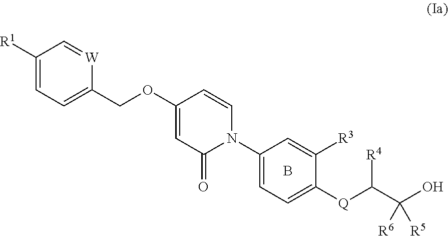
or a pharmaceutically acceptable salt thereof, wherein R¹ is a halogen or halogen-substitutedC₁-C₆ alkyl;
W is —N— or —CH—;
Q is —O—, —NH—, or —C—, or taken together with R⁴, aromatic ring B and R³ to form a heteroaryl;
R³ is a halogen, hydrogen, —OC₁-C₆alkyl, C₁-C₆alkyl, —O-halogen-substitutedC₁-C₆alkyl, halogen-substitutedC₁-C₆alkyl, cyano, SO₂C₁-C₆alkyl;
R⁴ is hydrogen, oxo, C₁-C₆alkyl, halogen-substitutedC₁-C₆alkyl or together with R⁶ form a C₃-C₆cycloalkyl or together with aromatic ring B, R³ and Q form a heteroaryl;
R⁵ and R⁶ are each independently selected from the group consisting of hydrogen, C₁-C₆alkyl, C₃-C₆cycloalkyl, C₁-C₆alkylC₃-C₆cycloalkyl, halogen-substitutedC₁-C₆alkyl, halogen-substitutedC₃-C₆cycloalkyl, or taken together form a C₃-C₆cycloalkyl.

16-23. (canceled)

24. A compound of claim 1, or a pharmaceutically acceptable salt thereof, selected from the group consisting of:
4-[(4-fluorobenzyl)oxy]-1-[4-(2-hydroxy-2-methylpropoxy)-3-
methylphenyl]pyridin-2(1H)-one;
1-[4-(2-cyclopropyl-2-hydroxyethoxy)phenyl]-4-[(4-fluoro-
benzyl)oxy]pyridin-2(1H)-one;
1-[4-(2-cyclopropyl-2-hydroxyethoxy)-3-methoxyphenyl]-4-[(4-
fluorobenzyl)oxy]pyridin-2(1H)-one;
1-[3-chloro-4-(2-hydroxy-2-methylpropoxy)phenyl]-4-[(4-
fluorobenzyl)oxy]pyridin-2(1H)-one;
4-[(4-fluorobenzyl)oxy]-1-[4-(2-hydroxy-2-methyl-
propoxy)phenyl]pyridin-2(1H)-one;
4-[(4-fluorobenzyl)oxy]-1-[4-(2-hydroxy-2-methylpropoxy)-
3-methoxyphenyl]pyridin-2(1H)-one;
4-[(4-fluorobenzyl)oxy]-1-[3-fluoro-4-(2-hydroxy-2-
methylpropoxy)phenyl]pyridin-2(1H)-one;
4-[(4-fluorobenzyl)oxy]-1-[4-(3-hydroxy-2,2-dimethyl-
propoxy)phenyl]pyridin-2(1H)-one;
4-[(4-fluorobenzyl)oxy]-1-[4-(3-hydroxy-2,2-dimethyl-
propoxy)-3-methoxyphenyl]pyridin-2(1H)-one;
4-(4-Fluorobenzyloxy)-1-[2-(2-hydroxypropan-2-yl)quinolin-6-
yl]pyridin-2-(1H)-one;
4-[(5-chloropyridin-2-yl)methoxy]-1-[4-(2-cyclopropyl-2-
hydroxyethoxy)-3-methoxyphenyl]pyridin-2(1H)-one;
4-[(5-chloropyridin-2-yl)methoxy]-1-[4-(2-cyclopropyl-2-
hydroxyethoxy)phenyl]pyridin-2(1H)-one;
1-[4-(2-cyclopropyl-2-hydroxyethoxy)phenyl]-4-[(3,4-
difluorobenzyl)oxy]pyridin-2(1H)-one;
1-{4-[(2R)-2-cyclopropyl-2-hydroxyethoxy]-3-methoxyphenyl}-
4-[(4-fluorobenzyl)oxy]pyridin-2(1H)-one;
1-{4-[(2R)-2-cyclopropyl-2-hydroxyethoxy]-3-methoxyphenyl}-
4-[(4-fluorobenzyl)oxy]pyridin-2(1H)-one;
1-{4-[(2S)-2-cyclopropyl-2-hydroxyethoxy]phenyl}-4-[(4-
fluorobenzyl)oxy]pyridin-2(1H)-one;
1-{4-[(2R)-2-cyclopropyl-2-hydroxyethoxy]phenyl}-4-[(4-
fluorobenzyl)oxy]pyridin-2(1H)-one;
methyl (2-fluoro-4-{4-[(4-fluorobenzyl)oxy]-2-oxopyridin-
1(2H)-yl}phenoxy)acetate
4-[(4-fluorobenzyl)oxy]-1-{4-[(1-hydroxycyclopropyl)methoxy]-
3-methylphenyl}pyridin-2(1H)-one;
propan-2-yl (4-{4-[(4-fluorobenzyl)oxy]-2-oxopyridin-1(2H)-
yl}-2-methylphenoxy)acetate;
methyl (4-{4-[(4-fluorobenzyl)oxy]-2-oxopyridin-1(2H)-yl}-
2-methylphenoxy)acetate;
methyl (4-{4-[(4-fluorobenzyl)oxy]-2-oxopyridin-1(2H)-yl}-
2-methoxyphenoxy)acetate;
4-[(4-fluorobenzyl)oxy]-1-[4-(3-hydroxy-3-methylbutoxy)-3-
methoxyphenyl]pyridin-2(1H)-one;
4-[(5-chloropyridin-2-yl)methoxy]-1-{4-[(2R)-2-cyclopropyl-
2-hydroxyethoxy]-3-methoxyphenyl}pyridin-2(1H)-one;
4-[(5-chloropyridin-2-yl)methoxy]-1-{4-[(2R)-2-cyclopropyl-
2-hydroxyethoxy]phenyl}pyridin-2(1H)-one;
1-{4-[(2R)-2-cyclopropyl-2-hydroxyethoxy]phenyl}-4-[(3,4-
difluorobenzyl)oxy]pyridin-2(1H)-one;
4-[(5-chloropyridin-2-yl)methoxy]-1-{4-[(2S)-2-cyclopropyl-
2-hydroxyethoxy]-3-methoxyphenyl}pyridin-2(1H)-one;
1-{4-[(2S)-2-cyclopropyl-2-hydroxyethoxy]phenyl}-4-[(3,4-
difluorobenzyl)oxy]pyridin-2(1H)-one;
4-[(5-chloropyridin-2-yl)methoxy]-1-(4-[(2S)-2-cyclopropyl-
2-hydroxyethoxy]phenyl}pyridin-2(1H)-one;
1-(3-chloro-4-[(1-hydroxycyclopropyl)methoxy]phenyl}-4-[(4-
fluorobenzyl)oxy]pyridin-2(1H)-one;
4-[(4-fluorobenzyl)oxy]-1-{3-fluoro-4-[(1-hydroxy-
cyclopropyl)methoxy]phenyl}pyridin-2(1H)-one;
4-[(4-fluorobenzyl)oxy]-1-{4-[(1-hydroxycyclopropyl)methoxy]-
3-methoxyphenyl}pyridin-2(1H)-one;
1-{4-[(2S)-2-cyclopropyl-2-hydroxyethoxy]-3-methoxyphenyl}-
4-[(3,4-difluorobenzyl)oxy]pyridin-2(1H)-one; and
1-{4-[(2R)-2-cyclopropyl-2-hydroxyethoxy]-3-methoxyphenyl}-
4-[(3,4-difluorobenzyl)oxy]pyridin-2(1H)-one.

25. A compound, or a pharmaceutically acceptable salt thereof, of claim 24 selected from the group consisting of:
4-[(4-Fluorobenzyloxy)]-1-[4-(2-hydroxy-2-methylpropoxy)-
3-methylphenyl]pyridin-2(1H)-one;
4-[(4-Fluorobenzyl)oxy]-1-{4-[(1-hydroxycyclopropyl)methoxy]-3-
methylphenyl}pyridin-2(1H)-one;
4-(4-Fluorobenzyloxy)-1-[2-(2-hydroxypropan-2-yl)quinolin-6-
yl]pyridin-2-(1H)-one;
1-[3-chloro-4-(2-hydroxy-2-methylpropoxy)phenyl]-4-[(4-
fluorobenzyl)oxy]pyridin-2(1H)-one;
4-[(5-chloropyridin-2-yl)methoxy]-1-[4-(2-cyclopropyl-2-
hydroxyethoxy)phenyl]pyridin-2(1H)-one;
4-[(5-chloropyridin-2-yl)methoxy]-1-{4-[(2R)-2-cyclopropyl-2-
hydroxyethoxy]phenyl}pyridin-2(1H)-one;
4-[(5-chloropyridin-2-yl)methoxy]-1-{4-[(2S)-2-cyclopropyl-2-
hydroxyethoxy]phenyl}pyridin-2(1H)-one; and
1-(3-chloro-4-[(1-hydroxycyclopropyl)methoxy]phenyl}-4-[(4-
fluorobenzyl)oxy]pyridin-2(1H)-one.

26. A pharmaceutical composition comprising a compound of claim 1, or a pharmaceutically acceptable salt thereof, and a pharmaceutically acceptable carrier.

27-28. (canceled)

29. A method for the treatment of a condition selected from the group consisting of obesity, diabetes, fatty liver, bulimia, depression, or anxiety comprising administering to an individual a pharmaceutical composition comprising the compound of claim 1.
